(19) United States
(12) Patent Application Publication
(10) Pub. No.: US 2026/0315628 A1
(43) Pub. Date: Oct. 15, 2026

(54) CSF1-R TARGETED MOLECULAR MRI AGENTS FOR NON-INVASIVE IMAGING OF NEURO- AND PERIPHERAL INFLAMMATION

(71) Applicants: Flavocure Biotech, Inc., Baltimore, MD (US); Baylor College of Medicine, Houston, TX (US)

(72) Inventors: Eric Tanifum, Richmond, TX (US); Ngeh J. Toyang, Elkridge, MD (US); Henry I. C. Lowe, Wellington, FL (US); Xianwei Sun, Houston, TX (US); Andrew Arun Badchhape, Houston, TX (US)

(73) Assignees: Flavocure Biotech, Inc., Baltimore, MD (US); Texas Children's Hospital, Houston, TX (US); Baylor College of Medicine, Houston, TX (US); Lothera LLC, Elkridge, MD (US)

(21) Appl. No.: 19/467,668

(22) Filed: Feb. 2, 2026

Related U.S. Application Data
(60) Provisional application No. 63/752,599, filed on Jan. 31, 2025.

(51) Int. Cl. A61K 49/18; A61K 49/08; A61K 49/10

(52) U.S. Cl. CPC A61K 49/1812; A61K 49/085; A61K 49/108

(57) ABSTRACT

CSF1-R targeted molecular MRI agents for non-invasive imaging of neuro- and peripheral inflammation are provided.

### CROSS-REFERENCE TO RELATED APPLICATIONS

This application claims priority from U.S. Provisional Patent Application No. 63/752,599, filed on Jan. 31, 2025, which is incorporated by reference herein in its entirety.

### BACKGROUND

Parkinson's disease (PD) is the fastest growing neurological disorder, affecting over 11.8 million individuals across the globe in 2021. In the United States, about 90,000 new cases are reported annually, representing a 50% increase from previous estimates, and leading to an estimated 1.2 million people living with the disease by 2030. PD is also highly heterogeneous, with complex pathogenesis that results in significant neuronal damage occurring before motor deficits appear. Because current diagnostic metrics focus on motor symptoms, clinical diagnosis is delayed relative to underlying pathophysiology. This complexity also limits the development of disease modifying treatments as no single biomarker captures the disease's multifaceted biology, and therapies aimed at one mechanism cannot overcome a disorder driven by many interacting pathways. Technologies that enable earlier detection and biomarker-guided stratification are required to accelerate the development of therapies that can alter the disease course.

Neuroinflammation mediated by microglia activation is increasingly recognized as a central and dynamic contributor to the pathogenesis of PD, making it a potential biomarker candidate. In prodromal and early stages of the disease, microglia may initially adopt a protective phenotype, clearing α-synuclein (α-syn) aggregates and releasing trophic factors. However, as the disease progresses, microglia shift toward a chronic pro-inflammatory state accompanied by reactive astrocytosis. Persistent glial activation accelerates neuronal death as accumulation of α-syn aggregates in the form of Lewy bodies and Lewy neurites spread from the medulla oblongata and olfactory systems through to the lower raphe nuclei and locus, to the substantia nigra pars compacta and amygdala, where the first clinical symptoms are triggered. Furthermore, persistent glial activation perpetuates recruitment of peripheral immune cells into the brain where they amplify neuroinflammation and contribute to disease progression by altering T-cell behavior, monocyte trafficking, and peripheral cytokine profiles. Taken together, these findings highlight microglia as early sensitive biomarkers of disease because they are the earliest and most dynamic responders to α-syn pathology, act as central amplifiers of neuroinflammation through cytokine signaling, and bridge innate and adaptive immunity. This suggests that noninvasive imaging tools that can profile microglia activity in vivo could enable earlier detection and patient stratification.

Positron emission tomography (PET) tracers, the current gold standard molecular imaging tools to noninvasively profile amyloid-O plaques and hyperphosphorylated tau tangles, have been immensely useful in Alzheimer's disease. However, such tools have not been adopted for routine clinical diagnosis or monitoring of neuroinflammation or Lewy pathology in PD. PET tracers of the mitochondrial translocator protein (TSPO) have provided in vivo evidence of microglial activation in PD patients, but variability in tracer specificity and genetic polymorphisms affecting TSPO binding have limited their use to research protocols. Alternative approaches including second- and third-generation TSPO tracers with improved signal-to-noise ratios and exploratory PET ligands targeting alternative neuroinflammatory pathways such as COX-2, P2X7 receptors, and the membrane bound protein, colony-stimulating factor 1 receptor (CSF1-R), are being actively pursued to overcome TSPO's limitations. Tracers targeting CSF1-R such as [¹¹C]CPPC and [¹⁸F]CPPC have shown promising preclinical and early clinical results, but challenges remain around optimizing brain penetration, selectivity, and low signal-to-noise for accurate noninvasive spatiotemporal routine profiling of microglia mediated neuroinflammation in the clinic.

Previously, in rodent models of neurodegenerative disorders, it was demonstrated that following intravenous administration (i.v.), liposomal nanoparticles bearing a magnetic resonance imaging (MRI) contrast payload and labeled with specific ligands passively cross into the brain parenchyma and label specific disease targets based on the targeting ligand. These tools enabled in vivo separation of treated disease cohorts from controls using noninvasive molecular magnetic resonance imaging (mMRI).

Recently, it was discovered that the flavonoid Caflanone is a potent inhibitor of CSF1-R. It was hypothesized that a Caflanone (or derivative)-labeled variant of an mMRI probe (NCDx) would enable selective microglial binding and trigger cell-specific receptor-mediated internalization of the probe. CSF1-R's high enrichment on CNS-resident myeloid cells and its dynamic upregulation during neuroinflammation could provide both specificity and disease-responsive uptake. Combined with the high-resolution of MRI, the probe could highlight brain regions where deposition of Lewy pathology is accompanied by microglial activation, enabling noninvasive separation of disease cohorts from controls.

### SUMMARY

In one aspect, a liposomal composition is provided, the liposomal composition comprising a first phospholipid; a sterically bulky excipient that is capable of stabilizing the liposomal composition; a second phospholipid that is derivatized with a first polymer; a macrocyclic gadolinium-based imaging agent; and a third phospholipid that is derivatized with a second polymer, the second polymer being conjugated to a targeting ligand, the targeting ligand being represented by:

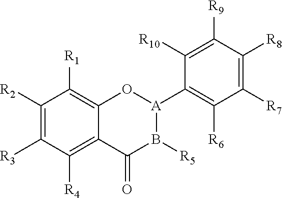

wherein R₁-R₁₀ may be any one or more substituents selected from a hydrogen molecule (H), a hydroxide group (OH), a methyl group (CH₃), a C₁-C₆ alkoxy group, a carboxyl group (COOH), a halide, an amino acid, a C₁-C₆ alkyl or alkenyl, or a C₁-C₆ alkyl amine, with the caveat that one of R₁ or R₃ must be either 3-methyl-2-butenyl or 3,7-dimethyl-2,6-octenyl, and the carbon-to-carbon A and B bond is either a single flavanone bond or a double flavone bond, or a salt thereof.

In an example aspect, the first phospholipid comprises hydrogenated soy L-a-phosphatidylcholine (“HSPC”); the sterically bulky excipient that is capable of stabilizing the liposomal composition comprises cholesterol (“Chol”); the second phospholipid that is derivatized with a first polymer comprises 1,2-distearoyl-sn-glycero-3-phosphoethanolamine-N-(methoxy (polyethylene glycol)-2000) (“DSPE-mPEG2000”); and the macrocyclic gadolinium-based imaging agent comprises gadolinium(3+) 2-[4,7,10-tris(carboxylatomethyl)-1,4,7,10-tetrazacyclododec-1-yl]acetate (“gadoterate” or “Gd(III)-DOTA”) and is conjugated to a fourth phospholipid, e.g.:

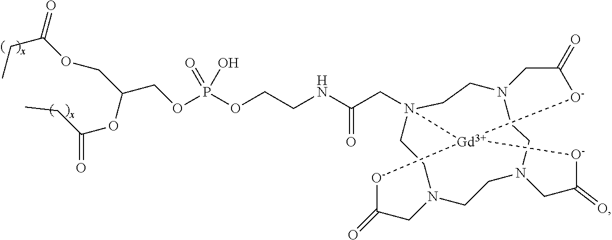

or a salt (e.g., a sodium salt) thereof. In some aspects, the variable x may be one of: 12, 13, 14, 15, 16, 17, or 18. In one aspect, the variable x is 16 (the conjugate: “Gd(III)-DOTA-DSPE”).

In some aspects, the third phospholipid that is derivatized with a second polymer may comprise:

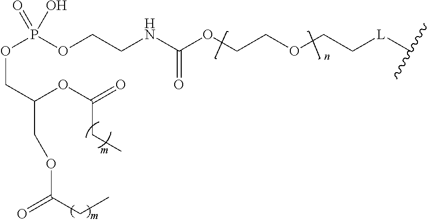

or a salt thereof. In some aspects, the variable n may be any integer from about 10 to about 100, for example, about 60 to about 100, about 70 to about 90, about 75 to about 85, about 77, or about 79. The variable m may be one of: 12, 13, 14, 15, 16, 17, or 18. For example, n may be 77, and m may be 14; n may be 79, and m may be 14; n may be 77, and m may be 16; and n may be 79, and m may be 16. The identity of “L” (the linking moiety between the third phospholipid that is derivatized with a second polymer and the targeting ligand) is quite variable and is typically a mere practical design choice for yield, purification, or scalability, with conservative variations having little or no effect on the effectiveness of the conjugate. In one aspect, L may comprise, e.g., one or more carbonyls (e.g., amido groups), an ether, including a polyether, and combinations of carbonyls and ethers. The teaching of specific “L” groups in this disclosure should not be considered as limiting, but, rather, quite the opposite: a mere brief selection to demonstrate a breadth of possibilities.

Generally, in one aspect, the targeting ligand comprises:

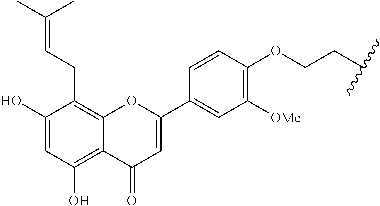 [SMILES: *CCOc1ccc(-c2cc(=O)c3c(O)cc(O)c(CC=C(C)C)c3o2)cc1OC]

In one example aspect, the targeting ligand is:

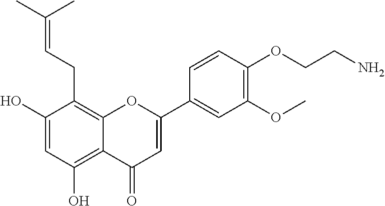 [SMILES: COc1cc(-c2cc(=O)c3c(O)cc(O)c(CC=C(C)C)c3o2)ccc1OCCN]

(“HLNT-06”) or a salt thereof.

In one such aspect, n is 77, m is 16, and the targeting ligand comprises:

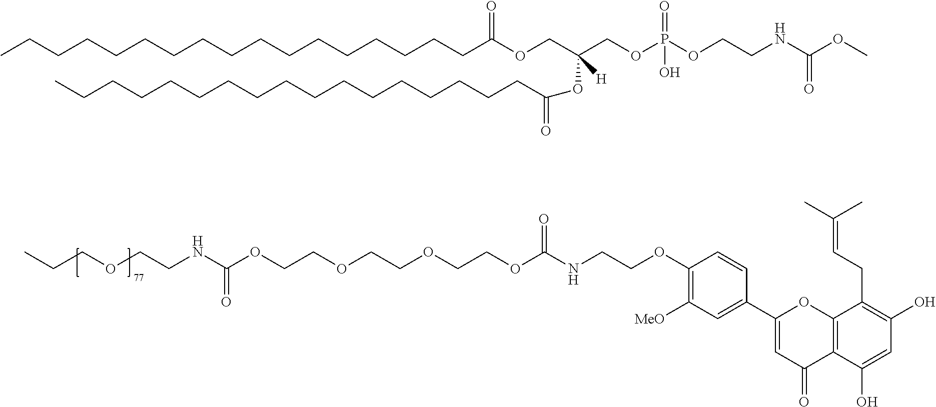

(“DSPE-PEG3400-Caflanone v.1”), including as a salt thereof. In such an aspect, “L” is:

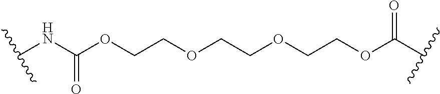 [SMILES: *NC(=O)OCCOCCOCCOC(*)=O]

DSPE-PEG3400-Caflanone v.1 was an early generation conjugate. It may be synthesized using HLNT-06, a 3-unit PEG linker, and DSPE-PEG-Amine.

In a further such aspect, n is 77, m is 16, and the targeting ligand is:

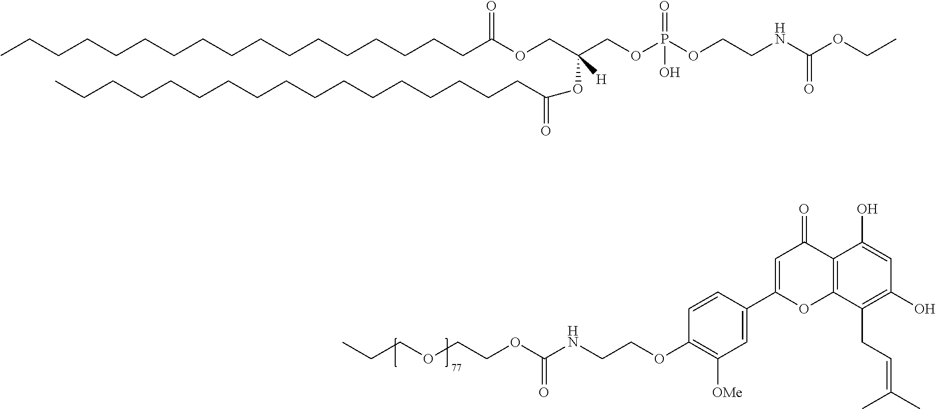

(“DSPE-PEG3400-Caflanone v.2”) including as a salt thereof. In such an aspect, “L” is:

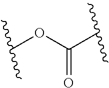 [SMILES: *OC(*)=O]

DSPE-PEG3400-Caflanone v.2 was synthesized from HLNT-06 and DSPE-PEG-OH. This approach provides high yields.

In yet a further such aspect, n is 77, m is 16, and the targeting ligand is:

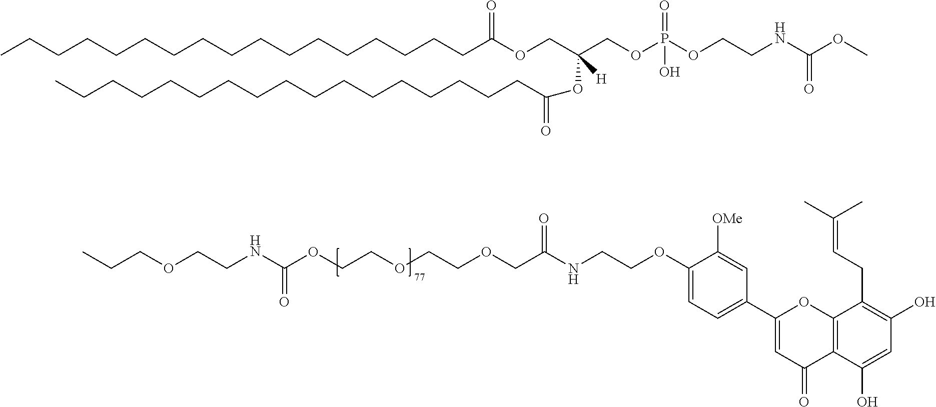

(“DSPE-PEG3400-Caflanone v.3”), including as a salt thereof. In such an aspect, “L” is:

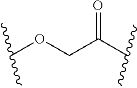 [SMILES: *OCC(*)=O]

DSPE-PEG3400-Caflanone v.3 was synthesized from HLNT-06 and DSPE-PEG-COOH. In this approach, the purification of the final product is simple and efficient.

In another example aspect, the targeting ligand is:

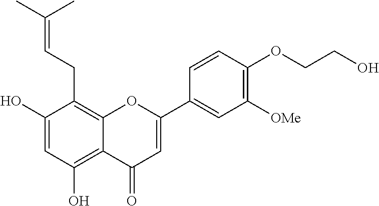 [SMILES: COc1cc(-c2cc(=O)c3c(O)cc(O)c(CC=C(C)C)c3o2)ccc1OCCO]

(“HLNT-EtOH”) or a salt thereof

In such an aspect, n is 77, m is 16, and the targeting ligand is:

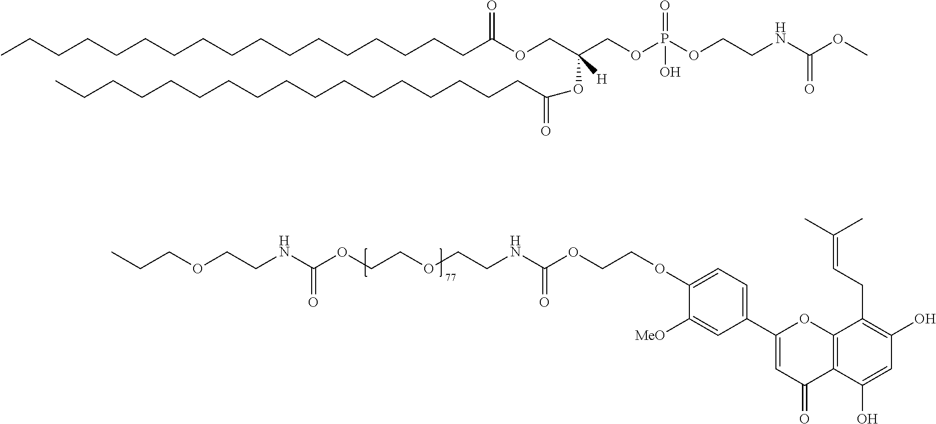

(“DSPE-PEG3400-Caflanone v.4”), including as a salt thereof. In such an aspect, “L” is:

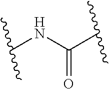 [SMILES: *NC(*)=O]

DSPE-PEG3400-Caflanone v.4 was synthesized and tested for scale-up purposes. It is synthesized from the hydroxyl analogue of HLNT-06 and DSPE-PEG-Amine. This approach provides high yields, and purification of the final product is simple and efficient.

In one aspect, a method for imaging activated microglia cells in a subject is provided. In one aspect, the activation is associated with cross-P sheet deposits. Although amyloid-β and α-syn deposits are used in the examples herein, the method may be applied to any protein that forms cross-B structures, such as tubulin associated unit (Tau), TAR DNA-binding protein 43 (TDP43), Transthyretin (TTR), huntingtin protein (HTT), and the like.

Indeed, as the method is keyed toward inflammation, rather than cross-β sheet deposits, per se, the method may be used to detect other microglia mediated inflammatory diseases that are not obviously or directly accompanied by protein aggregation, such as traumatic brain injury, PTSD, Dravet syndrome, and multiple sclerosis.

Further, the in vitro studies (microglia and macrophage cell lines) described herein suggest that NCDx can label both peripheral and intracranial myeloid cells and, therefore, demonstrates the potential to image both peripheral and intracranial inflammatory diseases.

The method may comprise introducing in the subject a detectable quantity of liposomal composition. The method may comprise allowing sufficient time for the liposomal composition to be associated with one or more activated microglia cells. The method may comprise detecting the liposomal composition associated with the one or more activated microglia cells. In one aspect, the method comprises detecting using magnetic resonance imaging (MRI). In one aspect, the method further includes aiding in the diagnosis of one or more neurodegenerative disease.

In one aspect, a method for imaging peripheral inflammation due to or associated with activated macrophages in a subject is provided. The method may comprise introducing in the subject a detectable quantity of liposomal composition. The method may comprise allowing sufficient time for the liposomal composition to be associated with the peripheral inflammation. The method may comprise detecting the liposomal composition associated with the peripheral inflammation.

In one aspect, the liposomal composition of the method for imaging activated microglia cells in a subject may comprise Gd(III)-DOTA-DSPE and DSPE-PEG3400-Caflanone (e.g., v.1-v.4 or another version having an optimized “L” group). In one aspect, the liposomal composition of the method for imaging activated microglia cells associated with cross-p sheet deposits in a subject may comprise HSPC, Chol, DSPE-mPEG2000, Gd(III)-DOTA-DSPE, and DSPE-PEG3400-Caflanone (e.g., v.1-v.4 or another version having an optimized “L” group).

In one aspect, a kit is provided, the kit comprising the components of any one of liposomal compositions described herein. In one aspect, the kit further comprises instructions for carrying out the methods as described herein.

### BRIEF DESCRIPTION OF THE FIGURES

FIG. 1 shows how PEGylated Caflanone interacts with the extracellular Ig domains of CSF1-R. (A) shows the molecular structure of Caflanone. (B) shows the molecular structure of PEGylated Caflanone (i.e., a derivative based on targeting ligand HLNT-06). (C) shows a schematic of the CSF1-R transmembrane receptor illustrating the intracellular kinase domain, the transmembrane domains, and the 5 extracellular Ig-like domains (D1-D5) with bound CSF1. (D) shows four druggable sites on the extracellular Ig-like domains with site 1 and site 2 on D2, site 3 on D5, and site 4 on D3. (E) and (F) show the predicted interaction of PEGylated Caflanone with CSF1-R when docked in site 3 or in site 2, respectively.

FIG. 2 shows the molecular components of an example of a caflanone-labeled mMRI probe, NCDx.

FIG. 3 shows an example schematic for the synthesis of HLNT-06.

FIG. 4 shows an example schematic for the synthesis of DSPE-PEG3400-caflanone v.1 from HLNT-06.

FIG. 5 shows the results of in vitro exposure of HMC-3 cells to DSPE-PEG3400-Caflanone v.1 liposomes. Signal is observed from the cytosolic compartment of the cells within 1.5 hours of exposure to the DSPE-PEG3400-Caflanone v.1 liposomes. Controls included cells treated with nanoparticles lacking the caflanone label and untreated cells.

FIG. 6 shows the results of in vitro exposure of SIM-A9 cells to DSPE-PEG3400-Caflanone v.1 liposomes. Signal is observed from the cytosolic compartment of the cells within 1.5 hours of exposure to the DSPE-PEG3400-Caflanone v.1 liposomes. Controls included cells treated with nanoparticles lacking the caflanone label and untreated cells.

FIG. 7 shows the results of in vivo MRI scans from a 12 month old APP/PSEN1 mouse (Tg) and age-matched wild type control (WT) after exposure to DSPE-PEG3400-Caflanone v.1 liposomes. The Tg mouse images show an increase in signal intensity post-treatment with the DSPE-PEG3400-Caflanone v.1 liposomes.

FIG. 8 shows a comparison of the change in signal intensity within the highlighted regions of interest in the cerebral cortex and hippocampal area between Tg animals and WT controls, demonstrating a statistically significant percent change.

FIG. 9 shows the results of ex-vivo immunohistochemical analysis and demonstrates a correlation between signal from the DSPE-PEG3400-Caflanone v.1 liposomes and IBA1 reactive cells surrounding amyloid β deposits in Tg animals. The signal occurs within the cytosolic compartment of the cells (arrows).

FIG. 10 shows an example schematic for the synthesis of DSPE-PEG3400-caflanone v.2 from HLNT-06.

FIG. 11 shows that murine and human microglia cell lines actively internalize NCDx (in this case, based on DSPE-PEG3400-caflanone v.2). (A) shows that SIM-A9 cells (murine microglia cell line) avidly internalize NCDx within 2 hours of exposure compared to controls. (B) shows that HMC-3 cells (human microglia cell line) also avidly uptake NCDx compared to controls. (C) shows that activation of SIM-A9 cells with CSF1 results in increased uptake of NCDx. (D) shows a plot of signal intensity per cell against exposure time and demonstrates that NCDx internalization exhibits saturation kinetics. (E) shows that activation of HMC-3 with CSF1 results in increased uptake of NCDx. (F) shows that NCDx uptake by HMC-3 cells exhibits saturation kinetics. (G) shows confocal microscopy images demonstrating that blocking the receptor binding sites with the free ligand inhibits NCDx uptake. (H) shows a plot of NCDx signal per cell co-incubated with the free ligand and demonstrates NCDx signal decay with increasing concentration of the free ligand. Scale bar=20 μm.

FIG. 12 shows in vivo contrast-enhanced MRI of mice injected with NCDx of caflanone-labeled particles administered to A53T and WT mice. Axial slices of the mouse (A) cortex and (B) olfactory bulb are shown for each tested animal on a jet colormap. Box and whisker plots for the (C) cortex and (D) olfactory bulb are shown as well. All images are set to same color scale. Scale bar=3 mm. Wilcoxon rank sum test: *p<0.05.

FIG. 13 shows ex vivo immunohistochemistry and demonstrates that in vivo accumulation of NCDx is driven primarily by microglia uptake. (A) shows brain tissue sections from treated TG mice and demonstrates NCDx signal in the cytosolic compartment of IBA-1 reactive cells. (B) shows that NCDx accumulation correlates with the presence of Lewy pathology. (C) demonstrates that NCDx signal does not show direct overlap or c-localization with GFAP reactivity. Scale bar=20 μm.

FIG. 14 shows an example schematic for the synthesis of HLNT-EtOH.

FIG. 15 shows an example schematic for the synthesis of DSPE-PEG3400-caflanone v.4 from HLNT-EtOH.

FIG. 16 shows NCDx uptake by RAW cells. (A) shows representative confocal images of NCDx uptake by RAW264.7 cells within 2 hours of exposure compared to controls. (B) shows CSF1 activated cells demonstrate an increase in uptake compared to non-activated cells. (C) shows NCDx kinetics, *P≤0.05, **P≤0.01, ***P≤0.001, and ****P≤0.0001. N=4 for all, one-way and two-way ANOVA was performed with Bonferroni's post hoc test and Tukey's test, respectively.

FIG. 17 provides example cross-sectional depictions of CSF1-R targeted liposomes in the form of. (A) a diagnostic variant (NCDx); (B) a treatment variant (NCThera); and (C) a fluorescent variant (NCFluor).

### DETAILED DESCRIPTION

The pathogenesis of PD has been shown to be driven by neuroinflammation with microglia as one of the key cellular contributors. The process is highly complex and heterogenous with spatiotemporal variability at different stages of the disease. This requires tools with high specificity for noninvasive profiling of the process in vivo. mMRI is uniquely suitable to address this problem because it can integrate the inherent high spatial resolution and soft-tissue contrast of conventional MRI with molecularly targeted contrast agents, enabling simultaneous acquisition of anatomical detail and functional/biological information about specific cellular or molecular processes at submillimeter isotropic resolution.

In this disclosure, it is demonstrated that polyethylene glycol (PEG)-tethered derivatives of Caflanone (PEGylated Caflanone) bind the extracellular domain of CSF1-R, and that NCDx actively labels murine and human microglia cell lines in vitro. It is further demonstrated that following i.v. administration in the A53T o-syn transgenic line M83 mouse model of PD., NCDx labels microglia in vivo, enabling noninvasive separation of pathology-bearing transgenic mice from wildtype controls. These in vivo data were validated by ex-vivo immunohistochemistry (IHC). The findings demonstrate that NCDx, has the potential to target microglia as a biomarker, and enable noninvasive separation of, e.g., parkinsonian brains, from controls in vivo, using MRI.

Thus, in one aspect, a novel mMRI probe is provided, the probe designed to specifically target colony stimulating factor-1 receptor (CSF1-R), expressed primarily on microglia in the brain. In silico data show that the targeting ligand binds the extracellular Ig domain of the receptor. In vitro cell uptake studies with murine and human microglia cell lines show that the probe binds the receptor, triggering active cell uptake, and in vivo MRI in the A53T mouse model of Parkinson's disease enabled effective separation of disease mice from controls. Ex-vivo immunohistochemical analysis (IHC) showed signal from the probe largely in the cytosolic compartment of IBA-1 reactive cells, confirming that the observed in vivo MRI signal in disease is due primarily to retention of the agent by microglia.

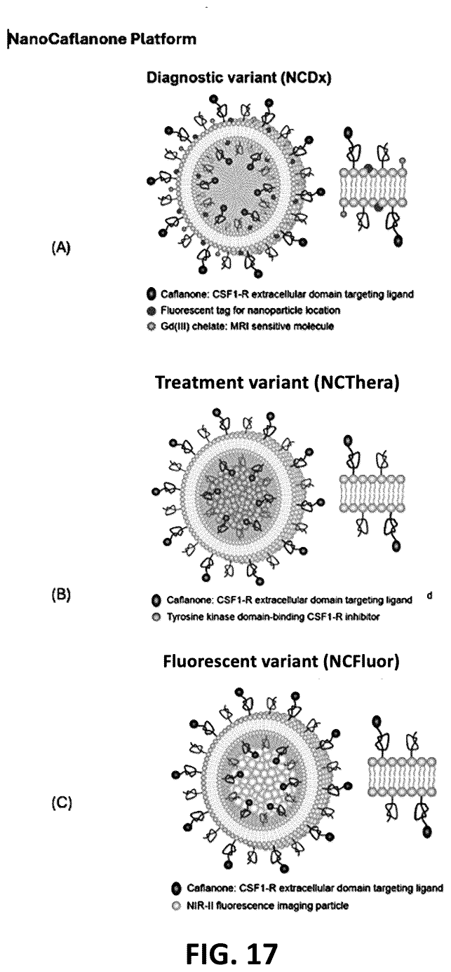

Thus, a novel molecular imaging technique for neuroinflammation by targeting CSF1-R with a liposomal composition bearing an MRI contrast payload is provided. The individual liposomes of the liposomal composition may be generally understood as depicted in cross-section form in FIG. 17(A).

In one aspect, the liposomal composition comprises a first phospholipid; a sterically bulky excipient that is capable of stabilizing the liposomal composition; a second phospholipid that is derivatized with a first polymer; a macrocyclic gadolinium-based imaging agent; and a third phospholipid that is derivatized with a second polymer, the second polymer being conjugated to a targeting ligand. The macrocyclic gadolinium-based imaging agent may be conjugated to a fourth phospholipid.

### Phospholipids

In some aspects, suitable phospholipids include those where the two hydrocarbon chains are between about 14 and about 24 carbon atoms in length and have varying degrees of unsaturation. In some aspects, suitable phospholipids include HSPC, 1,2-dipalmitoyl-sn-glycero-3-phosphocholine (“DPPC”), 1,2-distearoyl-sn-glycero-3-phosphocholine (“DSPC”), 1,2-distearoyl-sn-glycero-3-phosphoethanolamine (“DSPE”), and mixtures of two or more thereof. Suitable phospholipids may be naturally occurring or synthetic.

In some aspects, suitable phospholipids may include any of those listed in WO2005107820A1, the content of paragraphs [0031]-[0033] of which is incorporated by reference herein in its entirety.

### Polymer-Derivatized Phospholipids

In some aspects, the liposomes of the liposomal composition may include a surface that contains or is coated with flexible water soluble (hydrophilic) polymer chains. These polymer chains may prevent interaction between the liposomes and blood plasma components, the plasma components playing a role in uptake of liposomes by cells of the blood and removal of the liposomes from the blood. The liposomes may avoid uptake by the organs of the mononuclear phagocyte system, primarily the liver and spleen (the reticuloendothelial system).

In one aspect, the polymer in the derivatized phospholipid may be polyethylene glycol (“PEG”). The PEG may have any of a variety of molecular weights. In one example, the PEG chain may have a molecular weight between about 1,000-10,000 daltons. Once a liposome is formed, the PEG chains may provide a surface coating of hydrophilic chains sufficient to extend the blood circulation time of the liposomes in the absence of such a coating.

In some aspects, the second phospholipid that is derivatized with a first polymer comprises DSPE-mPEG2000. In some aspects, the third phospholipid that is derivatized with a second polymer comprises:

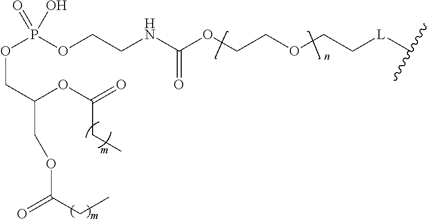

or a salt (e.g., an ammonium phosphate salt) thereof, wherein the variable n may be any integer from about 10 to about 100, for example, about 60 to about 100, about 70 to about 90, about 75 to about 85, about 77, or about 79. The variable m may be one of: 12, 13, 14, 15, 16, 17, or 18. For example, n may be 77, and m may be 14; n may be 79, and m may be 14; n may be 77, and m may be 16; and n may be 79, and m may be 16. L may be quite variable and may comprise, e.g., one or more carbonyls (e.g., amido groups), an ether, including a polyether, and combinations of amido groups and ethers. In some aspects, the third phospholipid that is derivatized with a second polymer comprises DSPE-PEG3400.

In some aspects, suitable polymers may include any of those listed in WO2005107820A1, the content of paragraphs [0034]-[0038] of which is incorporated by reference herein in its entirety. In some aspects, the phospholipid derivatized by a polymer may be any of those combinations disclosed in WO2016057812A1.

### Sterically Bulky Excipients

In some aspects, the liposomes may include stabilizing excipients. For example, the liposomal compositions may be formulated to comprise Chol. In other aspects, the liposomal compositions may comprise fatty alcohols, fatty acids, cholesterol esters, other pharmaceutically acceptable excipients, and mixtures thereof.

### Macrocyclic Gadolinium-based Imaging Agents

The liposomal composition comprises a macrocyclic Gd-based imaging agent. In some aspects, the macrocyclic gadolinium-based imaging agent comprises Gd(III)-DOTA conjugated to a phospholipid, e.g.:

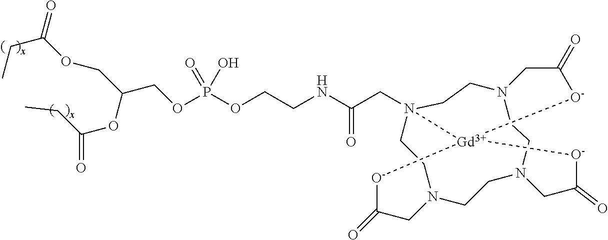

or a salt (e.g., a sodium salt) thereof. In some aspects, the variable x may be one of: 12, 13, 14, 15, 16, 17, or 18. In one aspect, the variable x is 16 and the conjugate is Gd(III)-DOTA-DSPE.

### Targeting Ligands

The liposome compositions comprise at least one phospholipid that is derivatized with a polymer, the polymer being conjugated to a targeting ligand. Thus, in some aspects, the phospholipid is modified to include a spacer chain. The spacer chain may be a hydrophilic polymer. The hydrophilic polymer may typically be end-functionalized for coupling to the targeting ligand. The functionalized end group may be, for example, a maleimide group, a bromoacetamide group, a disulfide group, an activated ester, or an aldehyde group. Hydrazide groups are reactive toward aldehydes, which may be generated on numerous biologically relevant compounds. Hydrazides may also be acylated by active esters or carbodiimide-activated carboxyl groups. Acyl azide groups reactive as acylating species may be easily obtained from hydrazides and permit the attachment of amino containing ligands.

In some aspects, the targeting ligand may be accessible from the surface of the liposome and may specifically bind or attach to, for example, one or more molecules or antigens. These targeting ligands may direct or target the liposomes to a specific cell or tissue, e.g., an α-syn aggregate and/or amyloid-β plaque, and may bind to a molecule or antigen on or associated with the cell or tissue.

In one aspect, the targeting ligand is represented by:

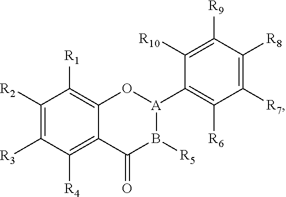

wherein R₁-R₁₀ may be any one or more substituents selected from a hydrogen molecule (H), a hydroxide group (OH), a methyl group (CH₃), a C₁-C₆ alkoxy group, a carboxyl group (COOH), a halide, an amino acid, a C₁-C₆ alkyl or alkenyl, or a C₁-C₆ alkyl amine, with the caveat that one of R₁ or R₃ must be either 3-methyl-2-butenyl or 3,7-dimethyl-2,6-octenyl, and the carbon-to-carbon A and B bond is either a single flavanone bond or a double flavone bond, or a salt thereof.

In one aspect, the targeting ligand is HLNT-06, HLNT-EtOH, or a salt thereof. In one aspect, the phospholipid-polymer-targeting ligand conjugate is DSPE-PEG3400-Caflanone (e.g., v.1-v.4 or another version having an optimized “L” group) or a salt thereof.

### Liposomes

“Liposomes” generally refer to spherical or roughly spherical particles containing an internal cavity. The walls of liposomes may include a bilayer of lipids. These lipids can be phospholipids. Numerous lipids and/or phospholipids may be used to make liposomes. One example are amphipathic lipids having hydrophobic and polar head group moieties, which may form spontaneously into bilayer vesicles in water, as exemplified by phospholipids, or which may be stably incorporated into lipid bilayers, with their hydrophobic moiety in contact with the interior, hydrophobic region of the bilayer membrane, and their polar head group moiety oriented toward the exterior, polar surface of the membrane. Liposomes may be prepared by any known method, including as described in the Examples herein, in WO2016057812A1, and in WO2012139080A1, which is incorporated by reference herein in its entirety. FIG. 17 provides an example cross-sectional depiction of a liposome comprising, e.g., a targeted contrast agent for MRI of activated microglia cells.

In one aspect, the DSPE-PEG3400-Caflanone liposomes comprise: HSPC; Chol; DSPE-mPEG2000; DSPE-PEG3400-Caflanone (e.g., v.1-v.4 or another version having an optimized “L” group); and Gd(III)-DOTA-DSPE. In some aspects, the first phospholipid may comprise DPPC, DSPC, or a mixture of DPPC and DSPC. In one aspect, the lipid composition and molar ratio (%) of components in the DSPE-PEG3400-Caflanone liposomes are HSPC:Chol:DSPE-mPEG2000:Gd(III)-DOTA-DSPE:DSPE-PEG3400-Caflanone=about 32: about 40: about 2.5: about 25: about 0.5. In some aspects, the molar ratio of any one of HSPC:Chol:DSPE-mPEG2000:Gd(III)-DOTA-DSPE:DSPE-PEG3400-Caflanone may be adjusted by up to 10%, thus, 32±10%: 40±10%: 2.5±20%: 25±10%: 0.5±10%. The molar ratio set forth here may change depending on the use of the liposome (e.g., what the liposome is carrying or targeting) and should be viewed as merely representative.

In a further aspect, the liposomes have vesicle size (Z-average) as measured by dynamic light scattering of less than 250 nm (D₅₀), including less than 200 nm (D₅₀), including about 180 nm (D₅₀), and including about 150 (D₅₀), and including about 120 nm (D₅₀).

Although the disclosure has focused on NCDx as a diagnostic, a skilled person will readily envision the use of the platform for other purposes. To this end, FIG. 17 provides example cross-sectional depictions of CSF1-R targeted liposomes in the form of: (A) a diagnostic variant (NCDx); (B) a treatment variant (NCThera); and (C) a fluorescent variant (NCFluor). It is contemplated that in the case of NCThera, one or more active pharmaceutical ingredients, e.g., an immunotherapy or gemcitabine, may serve a payload or co-payload.

The term “about” in conjunction with a number is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

### EXAMPLES

CSF1-R targeted liposomes with DSPE-PEG-Caflanone derivatives as the targeting moiety, Gd(III)-DSPE-DOTA as the MRI contrast source, and labeled with DiLC dye for particle tracking were formulated using standard protocols. In vitro nanoparticle cell uptake studies and blocking experiments with free Caflanone employing both human and murine microglia cell lines were used to establish receptor-mediated internalization. An AD mouse model (APP/PSEN1) was used for in vivo MRI studies. Mice were pre-scanned to establish a baseline followed by injection of the DSPE-PEG-Caflanone liposomes. Post-contrast scans were obtained at 4 days post-injection, followed by euthanasia, and brain removal for ex vivo immunohistochemical analysis.

Statistical analysis for the in vitro cell experiments was performed through one-way ANOVA with multiple pairwise comparisons between groups using Fisher's least significant difference (LSD) test. Statistical analysis of in vivo experimental data was performed through the Kruskal-Wallis method with multiple pairwise comparisons using the Bonferroni method.

All animal studies were conducted under study-specific protocols approved by the Institutional Animal Care and Use Committee (IACUC) at Baylor College of Medicine. In all animal experiments, 6 mice were used in each test or control group.

### Example 1: In Silico Studies

### Protein and Ligand Preparation

The crystal structure of extracellular CSF-1R:CSF-1complex (PDB 4WRM1, resolution 6.85 Å) was preprocessed with Schrödinger Maestro 14.1. Preprocessing using the protein preparation wizard of Maestro involved filling in any missing side chains and loops, adding missing hydrogens, enumerating bond orders, determining protonation states for het groups at pH±2.0, optimizing h-bonds, followed by restrained minimization and deletion of waters molecules that are not in close proximity to het groups. Upon preparation of the protein, the quality of the prepared protein was assessed by running a protein reliability report, which showed no red flags, indicating the prepared protein is reliable for computational modeling.

PEGylated Caflanone was prepared using Maestro's LigPrep tool. That includes converting the 2D structure of the ligands to 3D structures, adding hydrogen atoms, determining partial charges of atoms, and generating multiple conformers and ionization states at pH 7.4±2.0 of the ligands.

### Site Identification for Extracellular Binding of Ligand

A less explored region of the CSF-1R domain is the extracellular immunoglobulin-like domains (Ig D1-D5). There was no reported 3D structure of CSF-1RD1-D5 with a crystalized drug or small molecule. Hence, an exploration was launched of a possible druggable binding site across CSF-1RD1-D5 using the SiteMap Maestro tool. SiteMap reported up to five top sites, with at least 15 site points per site. SiteScores (SScore) and druggability Scores (DScore) were generated and used to rank the sites.

### Docking

A receptor grid was generated using the site points reported from the potential druggable sites identified by SiteMap. The coordinates of the receptor grid were expanded to X, Y, Z of 15 A at all sites for docking PEGylated Caflanone due to the larger size of the molecule. All other default parameters were retained. Glide was used to dock the prepared PEGylated Caflanone ligand conformers using extra precision (XP) docking. Intramolecular H-bond was rewarded and the planarity of conjugated 7 groups enhanced. Glide scores that were generated were used to rank the best pose of the ligand when docked within the top ranked selected potential binding sites.

### Results

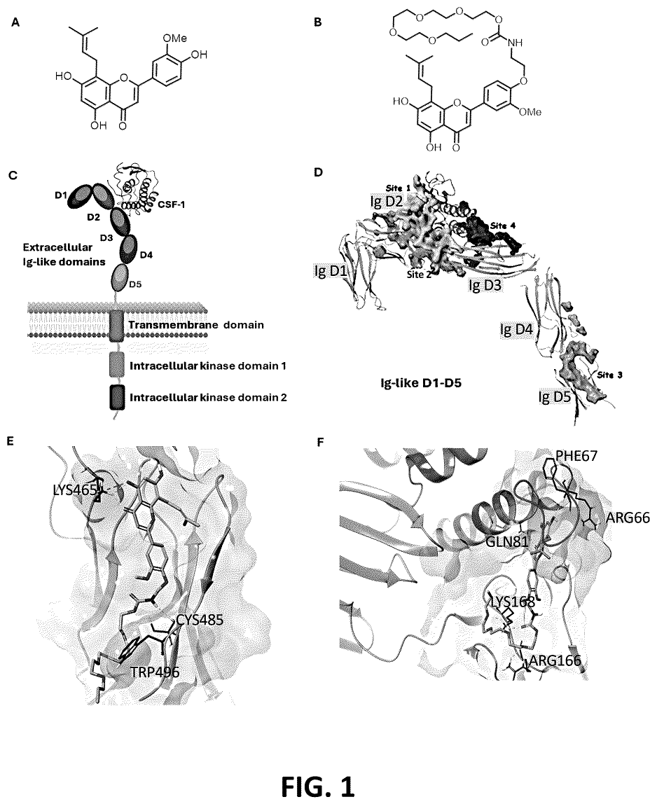

A PEGylated analogue of Caflanone binds the extracellular domain of CSF1-R. The targeting ligand is expressed on a PEG chain. Therefore, it was first established that a PEGylated analogue of Caflanone (FIG. 1(A-B)) has a suitable binding site on the extracellular domain of CSF1-R (FIG. 1(C)) using molecular simulation studies. Sites on the extracellular domain were prioritized because the nanoparticle requires specific surface interactions with the target cells for optimal efficacy. Specifically, possible druggable binding pockets on the extracellular immunoglobin (Ig)-like domain of CSF1-R were explored using the Schrödinger tool, SiteMap. SiteMap is an algorithm that identifies and ranks druggable sites on a protein using a SiteScore and a druggability Score (DScore). This resulted in four potential binding sites (FIG. 1(D)), including Site 1 (SiteScore of 0.920), Site 2 (SiteScore of 0.912), Site 3 (SiteScore of 0.916), and Site 4 (SiteScore of 0.913). Using molecular simulation docking studies, two sites (sites 2 and 3) emerged as potential high affinity binding sites to the molecule. Site 3, located on Ig D5, had the lowest predicted binding energy of −8.569 kcal/mol, indicating high affinity selective binding to the site (FIG. 1(E)). In this scenario, the construct extended the surface of the Ig D5 region with the amphiphilic PEG group traversing a narrow hydrophobic tunnel in which the nitrogen of the carbamate linker made H-bond interactions with the Cys 485 backbone and another H-bond between an oxygen atom in the PEG unit and Trp 496 residue. The flavonoid core structure on the other end showed H-bonds and a salt-bridge interaction with Lys 465 residue, extending the conformation of the construct and further anchoring it to the site. Site 2 (FIG. 1(F)), located between the Ig D2-D3 interface and close to the binding site of the natural ligand (CSF1), had the second best glide score of −7.132 kcal/mol. In this scenario, the PEG group interacted with a more solvent-exposed region of Ig D2, forming H-bonds with the residues of Arg 166 and Lys 168, resulting in a 1090 angular bend of PEG at C-1 of the carbamate. The flavonoid core extended into a hydrophobic cavity, making H-bonds with the backbone of Arg 66 and Phe 67, and multiple H-bonds with Gln 81.

### Example 2: CSF1-R Targeted mMRI Probe (NCDx) Design Comprising Four Components

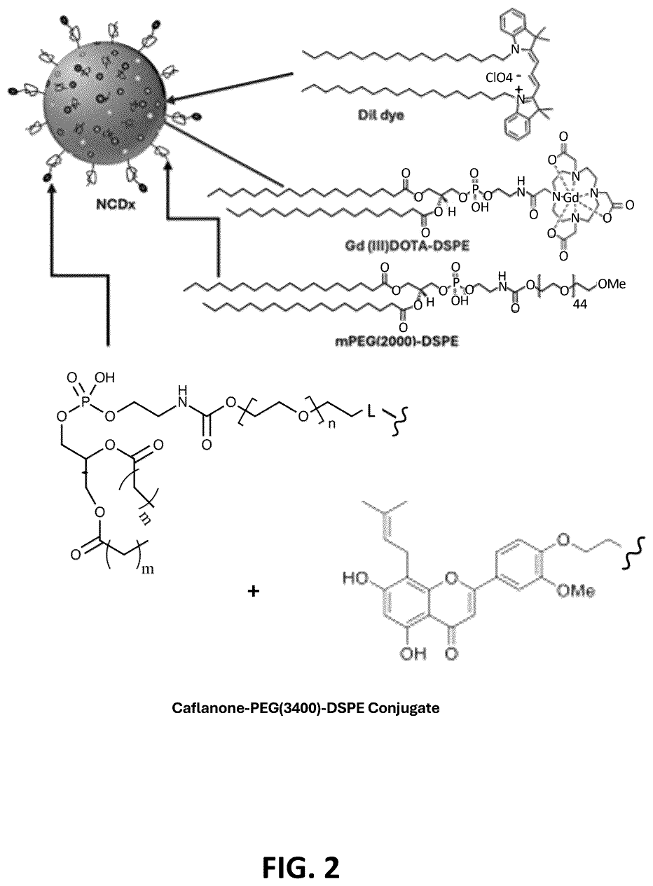

Based on the in silico data showing that PEGylated Caflanone binds the extracellular Ig-like domain of CSF1-R, a Caflanone labeled mMRI probe (NCDx) was designed and formulated. Chemical components (FIG. 2) include: (1) a novel Caflanone-PEG(3400)-DSPE conjugate (shown with a generalized “L” group with respective attachment points depicted by wavy lines) as the targeting moiety; (2) Gd(III)DOTA-DSPE as the MRI contrast source; (3) mPEG(2000)-DSPE; and (4) Commercial Dil dye as a fluorescent reporter on the probe.

### Example 3: Preparation of HLNT-06

### Synthesis of Compound 1

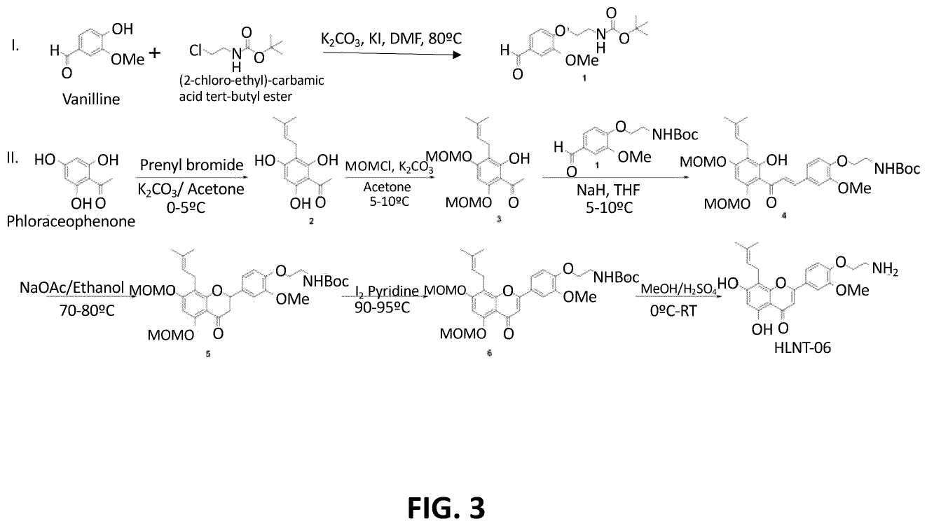

With reference to FIG. 3, vanillin (30.0 g, 0.19 mol), potassium carbonate (52.4 g, 0.38 mol), and potassium iodide (31.54 g, 0.19 mol) were suspended in DMF (300 ml), followed by the slow addition of (2-chloro-ethyl)-carbamic acid tert-butyl ester (81.46 g, 0.45 mol) in DMF (600 mL). The resultant mixture was stirred at 80° C. for 20 h and quenched with ice cold water (1500 mL). The product was extracted with ethyl acetate (3*1000 mL), washed with 1M NaCl (3*1000 mL), and concentrated under reduced pressure to obtain the compound 1 (62 g), which was used without further purification.

### Synthesis of Compound 2

Phloraceophenone (25 g, 0.148 mol) was dissolved in acetone (500 mL), followed by addition of K₂CO₃ (20.4 g, 0.148 mol) and prenyl bromide (15.4. g, 0.103 mol) at 0-5° C. The reaction mixture was stirred at this temperature for 2 hours, followed by filtration of solids. Solvent from the filtrate was evaporated under reduced pressure, and the residue purified by column chromatography eluted with ethyl acetate/hexanes gradient to afford the desired product 2 (10.9 g, yield 31%) as a white solid.

### Synthesis of Compound 3

Compound 2 (100.0 g, 0.42 mol) was taken in acetone (1500 mL), and K₂CO₃ (157.8 g, 1.14 mol) was added at 0-5° C. MOM-Cl (102.2 g, 1.26 mol) was added dropwise at 5-10° C., and the reaction temperature was slowly raised to room temperature and stirring continued for 3 h. The reaction mixture was filtered and washed with 400 mL of acetone. The filtrate was evaporated under reduced pressure, and the residue was redissolved in ethyl acetate, washed with water, brine, and dried over sodium sulphate. Solvent was removed under reduced pressure, and the residue purified by column chromatography eluted with ethyl acetate/hexanes gradient to afford the desired product 3 (60 g, yield 44%) as a yellow oil.

### Synthesis of Compound 4

Compound 3 (30 g, 0.09 mol) was dissolved in THF (300 mL) at 0-5° C., and NaH (4.32 g, 0.11 mol) was added and the resulting mixture stirred for 15 min, followed by dropwise addition of compound 1 (34.3 g, 0.11 mol) in THF (600 mL) at 5-10° C. Progress of the reaction was monitored by TLC and upon completion, it was quenched with DI-water (600 mL) and the product extracted with ethyl acetate (3*300 mL). The combined organic layers were concentrated and purified by column chromatography eluted with ethyl acetate/hexanes gradient to afford the desired product 4 (40 g, yield 74%) as yellow oil.

### Synthesis of Compound 5

Compound 4 (100 g, 0.16 mol) was suspended in 1000 mL of EtOH and 400 mL of DI-water, followed by addition of NaOAc (81.7 g, 0.99 mol) and heated to 70-80° C. for 4 hours. Progress of the reaction was monitored by TLC and upon completion, it was diluted with DI-water (1000 mL), and the product was extracted with ethyl acetate (3*500 mL). The combined ethyl acetate layers were dried over sodium sulphate and evaporated under reduced pressure to obtain a crude product which was purified by column chromatography eluted with ethyl acetate/hexanes gradient to afford the desired product 5 (35 g, yield 35%) as white solid.

### Synthesis of Compound 6

Compound 5 (35.0 g, 0.058 mol) was suspended in 500 mL of pyridine followed by addition of iodine (7.3 g, 0.058 mol) and the temperature raised to 90-95° C. for 8 h. Progress of the reaction was monitored by TLC. Upon completion, it was quenched with DI water (350 mL) and the pH was adjusted to 3-4 using citric acid. This was further diluted with 10% Na₂S₂O₃ solution and extracted with EtOAc. The organic layer was dried over sodium sulphate and the solvent removed under reduced pressure to get a crude product as yellow solid. This was purified by column chromatography eluted with ethyl acetate/hexanes gradient to afford the desired product 6 (24 g, yield 68%) a as yellow solid.

### Synthesis of HLNT-06

Compound 6 (24 g, 0.036 mol) was taken up in a mixture of methanol (24 mL) and THF (120 mL) followed by addition of conc. H₂SO₄ (24 mL) at 0° C. The reaction was allowed to warm to room temperature over 3 hours. It was then quenched by slow addition of ice-cold water and stirred for 30 min. The ensuing precipitate was filtered, washed with EtOAc, and dried under vacuum at 40° C. to get a yellow-colored crude product as a sulphate salt. The sulphate salt of HLNT-06 was basified using 10% sodium carbonate in water to obtain HLNT-06 (1Og, yield 67%) free base which was purified by heating in Methanol for 4 hours.

### Example 4: Synthesis DSPE-PEG3400-Caflanone v.1

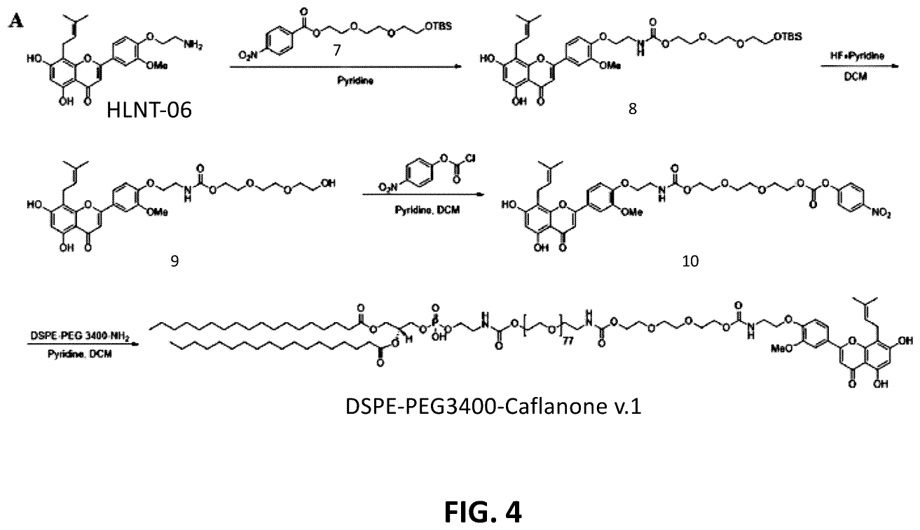

FIG. 4 shows an example schematic for the synthesis of the DSPE-PEG3400-Caflanone v.1. The active alcohol 3-unit PEG linker was prepared from the corresponding commercially available alcohol after two steps by standard methods. The free amine of HLNT-06 reacted with compound 7 to generate compound 8. The deprotection of the TBS with HF/Pry afforded compound 9. The free alcohol of compound 4 was activated by 4-nitrophenyl chloroformate to generated compound 10. Preparation of the DSPE-PEG3400-Caflanone v.1 proceeded by reacting compound 10 with DSPE-PEG3400-amine under mild conditions. The crude product was loaded into a 2000 MWCO dialysis bag and dialyzed against MES buffer (50 mM, 2×5 liters) for 8 h and then DI-water (3×5 liters) for 2d. After freeze drying, the desired compound DSPE-PEG3400-Caflanone v.1 was obtained.

### Example 5: Liposome Formulation with DSPE-PEG3400-Caflanone v.1

The formulation of control liposomes and target liposomes followed standard hydration/extrusion protocols. The lipid mixture (50 mM) comprising HSPC, DSPE-mPEG(2000), cholesterol, Gd(III)DSPE-DOTA (expressed on the lipid bilayer for MRI scanning), and the DSPE-PEG3400-Caflanone (presented on the surface of liposome and able to bind with CSF1-R and exhibit function in microglia cells; the control liposome did not comprise this compound) at a molar ratio of 32:2.5:40:25:0.5 in ethanol (600 μL) was stirred at 60-65° C. until all solids dissolved to form a clear solution. FIG. 2 depicts an example formulation of the liposomes prepared and tested herein. (As shown, the liposome bilayer incorporates DiLC dye for the purpose of fluorescence imaging.) 50 μL of DiLC dye was dissolved in ethanol (1 mg in 200 L) and added to the lipid solution, which was stirred for 5 min. 12 mL histidine-buffered saline (HBS) (10 mM histidine, 140 mM NaCl, ~pH 7.6) was added to the lipid solution at 60-65° C. and hydrated for 45 min. The hydrated lipid solution was extruded sequentially through 400 nm (5 passes) and 200 nm (8 passes) Nuclepore membranes at 60-65° C. using a high-pressure extruder (Northern Lipids, Vancouver, BC, Canada) to form liposomes of the desired size. The liposomal suspension was dialyzed against HBS using 300 kDa molecular weight cutoff membranes (Spectrum Laboratories Inc., CA, USA) to remove any unencapsulated lipids and ethanol. The hydrodynamic diameter of the liposomes was measured by DLS equipment to be 179.2+4.5 nm, and ICP-MS data showed a final lipid concentration of 85.3+2.3 mM.

### Example 6: Cell Uptake Studies with DSPE-PEG3400-Caflanone v.1

To verify the potential of cells to internalize target liposomes, in vitro cell experiments were performed. These studies included two cell lines: mouse microglia cell line SIM-A9 and human microglia cell line HMC-3. Cell only (UT) and cells incubated with control liposomes (that is, without DSPE-PEG3400-Caflanone in the liposome) were used as controls in the experiment.

The human microglial clone 3 cell line (HMC-3) was purchased from American Type Culture Collection (ATCC, Manassas, VA, USA). HMC-3 cells were cultured in Eagle's minimum essential media (EMEM, ATCC) supplemented with 10% FBS (Sigma-Aldrich, St. Louis, MO, USA) and 100 units/mL (U/mL) of penicillin/streptomycin (Invitrogen, Carlsbad, CA, USA), and incubated in a humidified atmosphere (5% CO₂) at 37° C.

SIM-A9 cells were purchased from ATCC, cultured in Dulbecco's Modified Eagle Medium: Nutrient Mixture F12 (DMEM/F12, Thermo Fisher Scientific, Waltham, MA, USA), supplemented with 10% fetal bovine serum (FBS, Thermo Fisher Scientific) and 100 units/mL (U/mL) penicillin/streptomycin (Invitrogen), and incubated in a humidified atmosphere (5% CO₂) at 37° C.

HMC-3 or SIM-A9 (2*104 cells/chamber) were seeded on 8 chamber culture slides (Falcon™ 354118) and allowed to adhere overnight. The cells were treated with liposomes (10 M lipid), which was diluted by medium. After 3 h of incubation, the supernatant was removed by pipetting, and the cells were washed with PBS three times. The cells were fixed in 4% paraformaldehyde for 15 min, washed with PBS three times, permeabilized by 0.1% Triton X-100 for 10 min, and washed again with PBS three times. The cells were blocked with 3% normal goat serum for 30 min. The supernatant was removed by pipetting and incubated with phalloidin 488 (1:100, ab176753, Abcam) for 1 h. This was followed by another wash with PBS and incubation with Hoechst (33342, Thermo Fisher Scientific) at rt for 7 min. Finally, the cells were washed with PBS three times, coverslipped, and imaged under an Olympus IX81 microscope using standard excitation/emission filters.

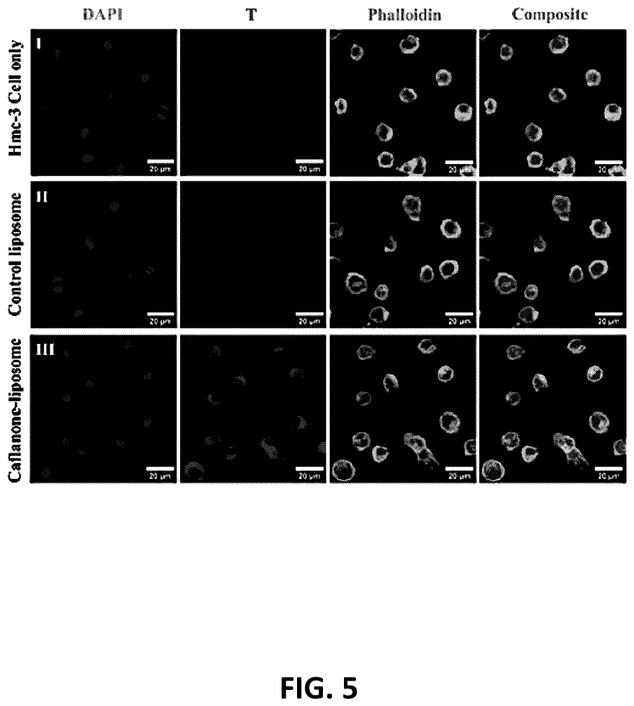

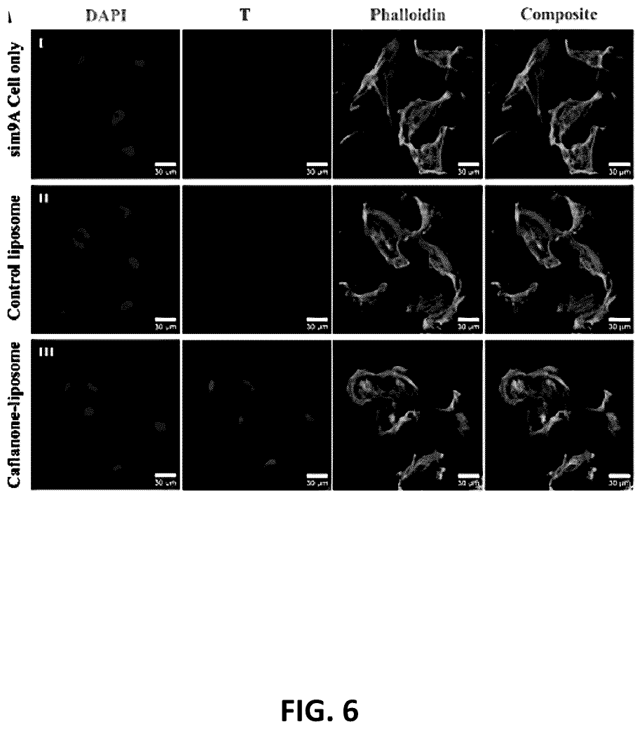

With reference to FIG. 5, HMC-3 cells incubated with the liposome composition present very strong interaction signal, while untreated HMC-3 cells and those incubated with control liposomes showed no signal at DiLC wavelength. Similarly, with reference to FIG. 6, untreated SIM-A9 cells and those incubated with control liposomes showed no noise or no apparent interaction signal at DiLC wavelength within 2 h of incubation, while SIM-A9 cells incubated with the DSPE-PEG-Caflanone liposomes present very strong interaction signal.

### Example 7: In Vivo MRI DSPE-PEG3400-Caflanone v.1

In vivo MRI studies show DSPE-PEG3400-Caflanone liposome accumulation in the brain. Mice were sedated using 3% isoflurane, placed on the MRI animal bed, and maintained at 1-2% isoflurane delivered using a nose cone. Breathing rate was monitored through a pressure pad placed below the abdomen. Mice underwent pre-contrast, baseline scans followed by intravenous administration of DSPE-PEG3400-Caflanone liposomes via the tail vein. Pre-contrast and delayed postcontrast MR images were acquired using a T1-weighted spin-echo (T1w-SE) sequence with the following parameters: TR=600 ms, TE=11.5 ms, slice thickness=1.2 mm, matrix=192×192, FOV=30 mm, slices=24, NEX=4. Coil calibration, RF calibration, and shimming were performed at the beginning of the study for each subject. At 4 days post-contrast administration, the animals were reimaged with the same scan parameters as in the baseline scans. Image analyses and signal quantification were performed with Osirix software (Pixmeo, Geneva, Switzerland). For quantitative signal analysis, signals from three ~1.2 mm thick coronal slices at bregma 0 (parietal-temporal lobe) were used to represent signal for the respective brain regions. Statistical analysis of in vivo experimental data was performed with a Student's T-Test.

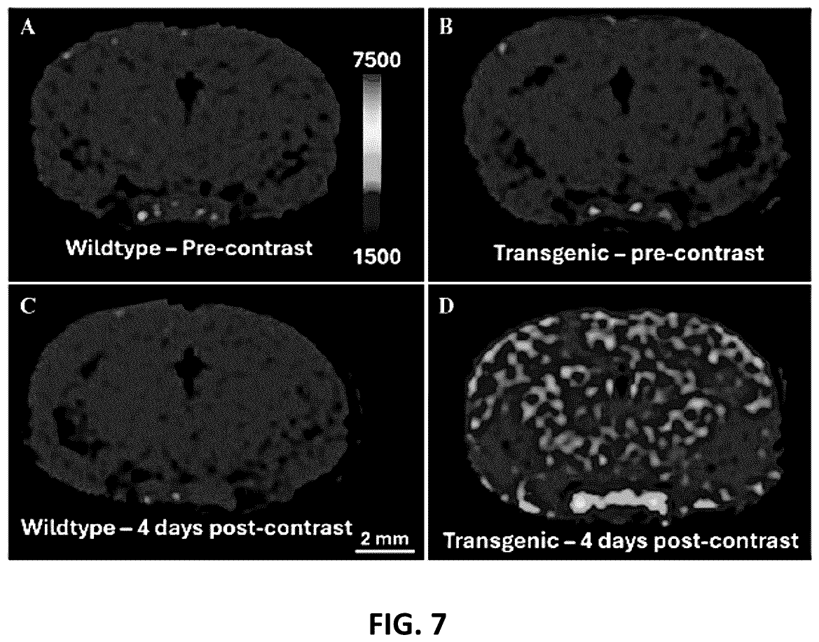

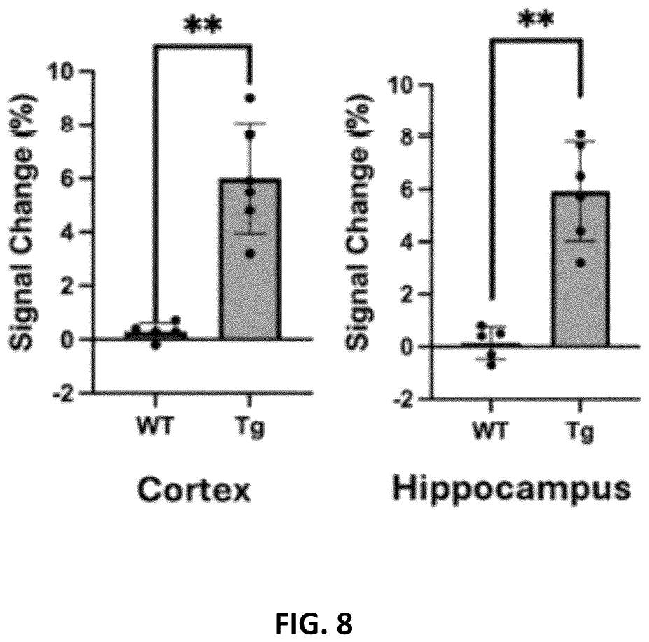

As shown in the pseudo-colored images from a 12-month-old mouse, signal (pre- and post-contrast scans) from each brain region was windowed similarly for both transgenic mice and wild type mice. The MR images show higher signal intensity in the post-contrast images of transgenic mice (FIG. 7(D)) compared to both pre-contrast scan (FIG. 7(B)) and wild type mice scan (FIGS. 7(A) and 7(C)). Signal change (%) between pre-contrast and delayed post-contrast images for each brain region was quantified by integration of signal in regions of interest shown by the white dotted lines. Box and whisker plots of the signal change in test animals show that post-contrast signal enhancement was seen in the cortical (+5.9%) and hippocampal (+6%) regions of transgenic mice administered T (FIG. 8). Wild type mice showed little to no enhancement in analogous regions. Mean differences in these regions of interest were significantly different between experimental groups (Student's T-test, p<0.005). A similar signal enhancement profile was observed in the brains of 9- to 6-month-old mice.

### Example 8: Correlations Between DSPE-PEG3400-Caflanone v.1 Liposome Signal, Amyloid-β, and IBA-1 Immunoreactivity

After the last MRI scan, the mice were perfused with PBS and 4% formalin, after which the brains were excised and stored in 30% sucrose solution until sectioning for histological analysis.

### Anti-IBA1 Staining:

The brains were embedded in Tissue-Tek O.C.T. and kept in liquid nitrogen for 30 min. The embedded tissue was sliced into 30 m thick sections with Lecia Biosystems Cryostats at −20° C. Immunofluorescence studies were performed as follows: tissue sections were washed twice with PBS, followed by antigen retrieval with citric acid buffer (pH~8.4)/microwave for 15 min. The sections were washed with PBS three times and permeabilized with 0.1% Triton-X 100 for 10 min, followed by washing with PBS (twice). The tissue was incubated with 10% normal goat serum at rt for 1 h, followed by incubation with a primary antibody, anti-IBA1 [Iba1/AIF-1 (E404W) XP® Rabbit mAb #17198, Cell Signaling Technology] (1:200 in 1% goat serum) and purified anti-β-amyloid [purified anti-β-amyloid 17-24 antibody, (4G8), host: mouse, Biolegend](1:200 in 1% goat serum) overnight at 4° C. The tissue was washed with PBS three times and incubated for 2 h at rt with Alexa Fluor 488 (anti-mouse) and Alexa Fluor 750 (anti-rabbit) secondary antibody (1:200 in PBS). After being washed with PBS two times, the tissue was incubated with Hoechst (33342, Thermo Fisher Scientific) at rt for 7 min. The sections were washed with PBS three times, coverslipped, and imaged under an Olympus IX81 microscope using standard excitation/emission filters.

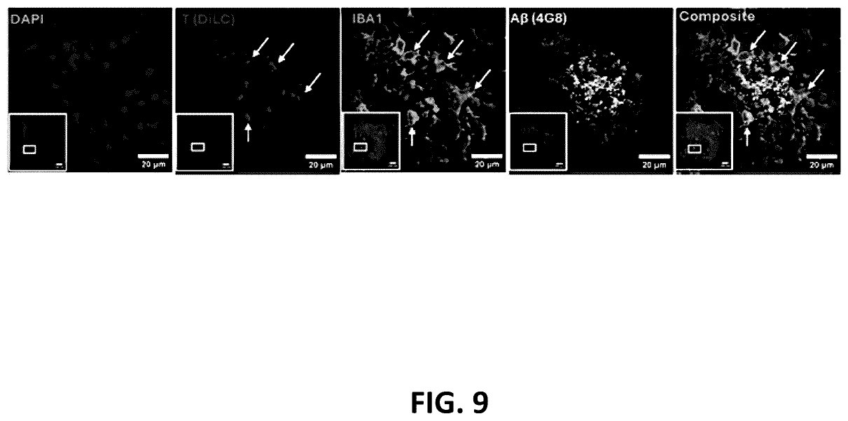

Microglia surround the amyloid beta plaques at the initial period of plaque formation and subsequently mount a harmful and non-resolving inflammatory response. Based on microglia surrounding the amyloid beta plaques and the in vivo MRI signal enhancement results, liposome composition accumulations in microglia by brain tissue sections stained with IBA-1 and amyloid beta antibody were assessed. 12-month-old mouse brain section showed that activated microglia cells surrounding the plaque that accumulated more liposomes gave a strong signal (FIG. 9). The composite image demonstrated that the liposomes accumulated inside of activated microglia cells, and inactivated microglia cells that are far away from plaques showed very little to no accumulation of liposomes.

### Example 9: Synthesis DSPE-PEG3400-Caflanone v.2 from HLNT-06

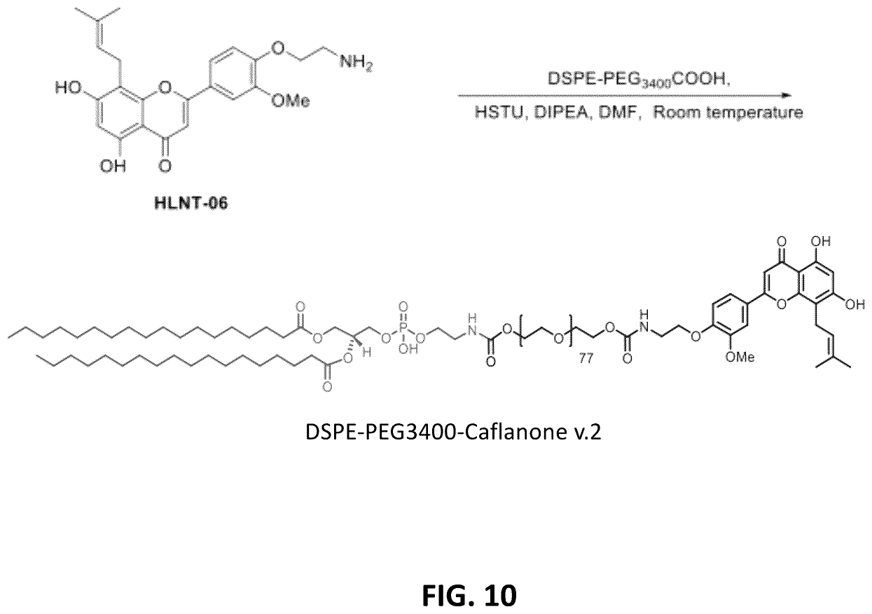

Referring to FIG. 10, to a solution of DSPE-PEG(3400)-COOH (500 mg, 0.1 mmol) and HLNT-06 (193 mg, 0.4 mmol) in dry DMF (8 mL) was added HSTU (160 mg, 0.4 mmol). The reaction mixture was stirred at room temperature for two days and concentrated under reduced pressure. The residue was diluted in methanol/water mixture (1:1, 8 ml), loaded into a 2000 MWCO dialysis bag, and dialyzed against MES buffer (50 mM, 2×5 liters) for 8 hours and then water (3×5 liters) for 2 days. The water was removed by freeze drying to obtain the desired compound as a yellow solid (253 mg, 47%).

### Example 10: Liposome Formulation with DSPE-PEG3400-Caflanone v.2

The formulation of control liposomes and target liposomes followed standard hydration/extrusion protocol. The lipid mixture containing HSPC, DSPE-mPEG(2000), cholesterol, Gd(III)DSPE-DOTA, and the caflanone-DSPE3.4k-conjugate v.2 (control liposome did not added this compound) at a molar ratio of 32:2.5:40:25:0.5 in ethanol (600 μL) was stirred at 60-65° C. until all solids dissolved to form a clear solution. 50 μL DiLC dye was added, dissolved in ethanol (1 mg in 200 μL), and stirred for 5 mins. 12 mL histidine-buffered saline (HBS) (10 mM histidine, 140 mM NaCl, ~pH 7.6) was added to the lipid solution at 60-65° C. and hydrated for 45 mins. The hydrated lipid solution was extruded sequentially through 400 nm (5 passes) and 200 nm (8 passes) Nuclepore membranes at 60-65° C. using a high-pressure extruder (Northern Lipids, Vancouver, BC, Canada) to form liposomes of the desired size. The liposomal suspension was dialyzed against histidine-buffered saline (HBS) using 300 kDa molecular weight cutoff membranes (Spectrum Laboratories Inc., CA, USA) to remove any unencapsulated lipids and ethanol. The nanoparticle size was determined by dynamic light scattering (DLS), and the concentrations of Gd(III) and total lipids in the final formulation were determined by inductively coupled plasma-optical emission spectrometry (ICP-OES).

The liposomes had a mean particle diameter of 155±4.5 nm (using Dynamic Light Scattering—DLS), and a total lipid molarity of 52.82±3.68 mM and Gd(III) concentration of 17.16±1.16 mM (using Inductively Coupled Plasma—ICP).

### Example 11: Cell Uptake Studies with DSPE-PEG3400-Caflanone v.2

### Cell Culture

SIM-A9 cells were purchased from American Tissue Culture Collection (ATCC, Manassas, VA, U.S.A.), cultured in Dulbecco's Modified Eagle Medium: Nutrient Mixture F12 (DMEM/F12, Thermo Fisher Scientific, Waltham, MA, U.S.A.) supplemented with 10% fetal bovine serum (FBS, Thermo Fisher Scientific) and 100 units/mL (U/mL) penicillin/streptomycin (Invitrogen, Carlsbad, CA, USA), and incubated in a humidified atmosphere (5% CO2) at 37° C.

The human microglial clone 3 cell line (HMC-3) was purchased from American Type Culture Collection (ATCC, Manassas, VA, USA). HMC-3 cells were cultured in Eagle's minimum essential media (EMEM, ATCC), supplemented with 10% FBS (Sigma-Aldrich, St. Louis, MO, USA) and 100 units/mL (U/mL) penicillin/streptomycin (Invitrogen, Carlsbad, CA, USA), and incubated in a humidified atmosphere (5% CO2) at 37° C.

### Cell Uptake Studies

HMC-3 or SIM-A9 (2*104 cells/chamber) were seeded on 8 chamber culture slides (Falcon™ 354118) and allowed to adhere overnight. The cells were treated with liposomes (10 μM lipid), which were diluted by medium. After 2 hours of incubation, the supernatant was removed by pipetting, and the cells were washed with PBS three times. The cells were fixed in 4% paraformaldehyde for 15 min and washed with PBS three times. The cells were permeabilized by 0.1% Triton X-100 for 10 min and washed with PBS three times. The cells were blocked with 3% normal goat serum for 30 min, and the supernatant was removed by pipetting and incubated with phalloidin 488 (1:100, ab176753, Abcam) for 1 hour. This was followed by another wash with PBS and incubation with Hoechst (33342, Thermo Fisher Scientific) at room temperature for 7 minutes. Finally, the cells were washed with PBS three times, coverslipped, and imaged under an Olympus IX81 microscope using standard excitation/emission filters.

### Results

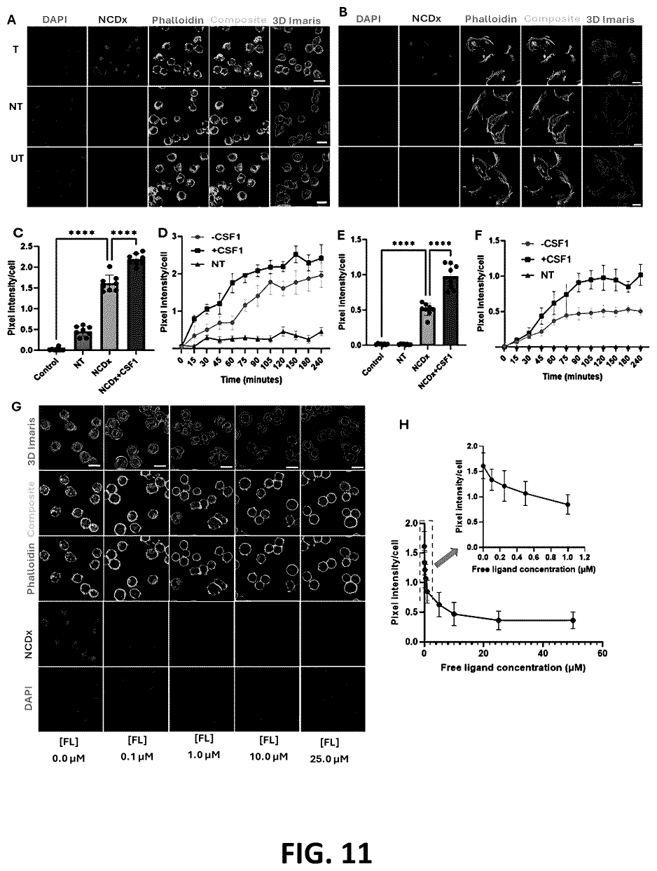

CSF1-R expressing cells actively internalize NCDx upon exposure to the probe. To verify that NCDx specifically interacts with CSF1-R expressing cells and triggers internalization, in vitro cell uptake experiments were performed using murine (SIM-A9) and human (HMC-3) microglia cell lines. Briefly, cells were incubated with NCDx (T) for 2 hours followed by a wash off to remove any uninternalized particles. The ensuing cells were mounted on slides imaged using confocal microscopy. Controls included cells treated with a nontargeted variant of the probe (NT) and untreated cells (UT). As exemplified by the images in FIG. 11(A), treated (T) SIM-A9 cells (nuclei seen in the DAPI channel) showed strong signal (NCDx) when viewed in the Dil channel. When signals from both channels are merged with signals from the Phaloidin channel, which maps out the cytoskeleton, the composite image shows the NCDx signal within the cytosol. The location of the nanoparticles is further accentuated by the 3D Imaris processed image, which unequivocally demonstrates that the NCDx signal is cytosolic. Neither the NT nor the UT controls showed any noteworthy signal in the Dil channel, suggesting that the targeting ligand (Caflanone) on NCDx plays a role in the internalization of the nanoparticles within two hours of exposure to SIM-A9 cells. HMC-3 (FIG. 11(B)) cells and the corresponding controls subjected to the same experimental conditions showed similar results. Murine and human primary astrocytes showed no NCDx internalization under similar conditions.

The effect of activation of each cell type with the natural substrate (CSF1), on NCDx uptake, was also assessed. As shown in FIG. 11(C), CSF1 activation results in a statistically significant increase in NCDx uptake by SIMA-9 cells compared to nonactivated cells. This data is corroborated by a kinetic study (FIG. 11(D)) assessed by change in pixel intensity per cell against exposure time, which shows an overall increase in uptake by the activated cells compared to the inactivated cells and the NT control. Activated HMC-3 cells (FIG. 11(E)) also show increased uptake of the agent and saturation kinetics (FIG. 11(F)) compared to the inactivated cells and controls. To establish specificity in particle uptake by the cells, a blocking study was performed in which ells were incubated with NCDx and increasing concentrations of the free ligand. As demonstrated by the representative confocal microscopy images of SIM-A9 cells in FIG. 11(G), increase in free ligand concentration results in diminished NCDx uptake. The full data is captured in a plot of signal intensity per cell against free ligand concentration (FIG. 11(H)), which shows that nanoparticle uptake bottoms out when the concentration of the free ligand reaches 20-25 μM. Taken together, these in vitro cell uptake data suggest that CSF1-R expressing cells actively internalize NCDx.

### Example 12: In Vivo MRI Using DSPE-PEG3400-Caflanone v.2 to Separate PD Transgenic Mice from Wild Types

### Animal Studies

### In Vivo MRI

All animal studies were conducted under a protocol approved by the Baylor College of Medicine Institutional Animal Care and Use Committee. In vivo MRI studies were performed in A53T transgenic mice (N=8, 12-14 months old) and age-matched wildtype controls (N=8) on a 1T permanent magnet (M2 system, Aspect Imaging, Shoham, Israel). For each imaging experiment, animals were sedated using 2-3% isoflurane and placed on a custom fabricated bed with an integrated face-cone for continuous anesthesia delivery by inhalation (1.5% isoflurane) for the duration of the experiment. Respiration rate was monitored by a pneumatically controlled pressure pad placed underneath the animal abdomen. To establish a baseline (pre-contrast), each mouse was subjected to an axial T1-weighted spin echo (T1w-SE) sequence with the following parameters: TR=600 ms, TE=11.5 ms, matrix=192×192, FOV=30 mm, slices=16, slice thickness=1.2 mm, NEX=2. Following the pre-contrast scans, mice were administered NCDx intravenously via tail vein at a dose of 0.1 mmol Gd(III)/kg and returned to their cages. Four days later, when the contrast had cleared systemic circulation, they were subjected to the same scan protocol using the same scan parameters to obtain post-contrast imaging data. Following this, each mouse was euthanized, perfused, and their brains excised and fixed for ex-vivo immunohistochemical analysis.

The raw imaging data was converted to dicoms and analyzed in Osirix (version 5.8.5, 64-bit). Region of interests (ROIs) were drawn in the cortex (bregma 0 mm) and olfactory bulbs (bregma 4-5 mm) of all animals in the pre-contrast and post-contrast scans. Each region was taken in three contiguous 1.2 mm slices. All images were analyzed on the same MR signal intensity scale (1500-8500 a.u.). Differences in subjects were analyzed as relative differences in signal change, which was calculated as the percentage increase in signal in the post-contrast scan relative to the baseline, pre-contrast scan (% Signal Change). All statistical analysis was performed using a non-parametric Wilcoxon rank-sum test (MATLAB, version 2025).

### Results

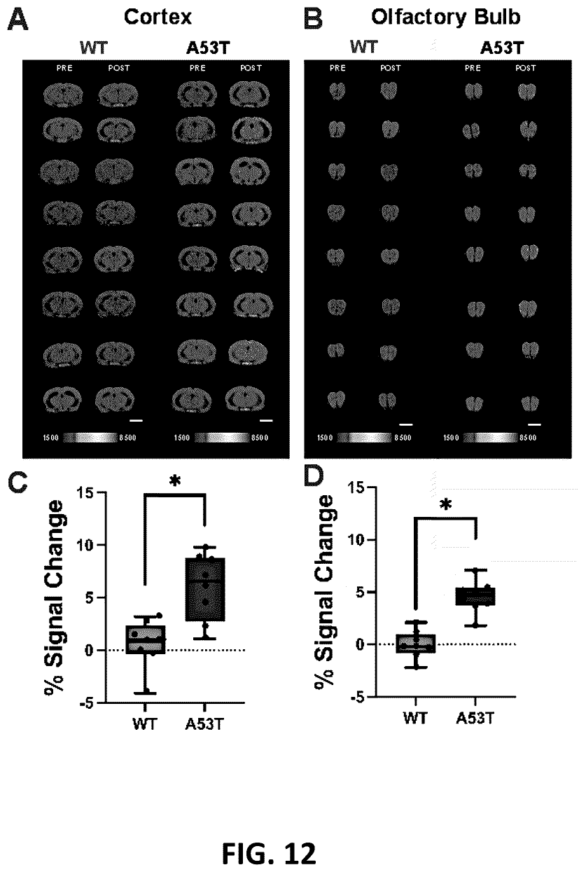

Conventional in vivo mMRI using NCDx enabled 80% separation of PD transgenic mice from wild types. Armed with strong in vitro data, the potential of NCDx to noninvasively separate Parkinsonian brains from controls was interrogated through a pilot study in a 12-14-month-old A53T α-synuclein (α-syn) transgenic line M83 PD mouse model. Transgenic mice (TG) in this cohort develop Lewy pathology in the medulla oblongata and olfactory systems, which subsequently spreads to the midbrain and eventually cortical areas in a time-related manner that parallels disease progression in humans. Mice were subjected to an axial T1-weighted spin echo (T1w-SE) scan sequence to obtain a baseline (pre-contrast) image, followed by injection of NCDx. Four days post-contrast administration, mice were subjected to the same scan protocol to obtain post-contrast images. Region of interests (ROIs) constituting three contiguous 1.2 mm slices drawn in the cortex (bregma 0 mm, FIG. 12(A)) and olfactory bulbs (bregma 4-5 mm, FIG. 12(B)) of all animals in the pre-contrast and post-contrast scans were analyzed for change in signal intensity. All images were analyzed on the same MR signal intensity scale (1500-8500 a.u.). 80% of the transgenic mice showed an increase in cortical signal in post-contrast images compared to the baseline (FIG. 12(C)). In slices obtained from the olfactory bulb (FIG. 12(D)), percent change in signal intensity between baseline and post-contrast scans suggested that 88% of the TG mice could be separated from controls.

### Radiomics

Post-contrast MRIs were first processed by registering a high-resolution brain atlas, annotated with 27 brain regions. This was followed by automatic extraction of cerebral cortex and olfactory bulb areas, using 3D Slicer. Radiomic features (RFs) were extracted using the Python package PyRadiomics(van Griethuysen et al., 2017), with and without applying filters (wavelet and Laplacian of Gaussian). Feature selection followed recommendations from the International Biomarker Standardization Initiative (IBSI)(Whybra et al., 2024), which identifies a subset of reproducible radiomic features suitable for research. RFs from cerebral cortex and/or olfactory bulb were examined in parallel. Although the same scanner and imaging protocol were used, and images were acquired within a similar time frame, a parallel analysis was performed to confirm the robustness of the findings. Images were resampled to isotropic resolution (0.15×0.15×0.15 mm), and pixel values were minmax normalized.

A standard machine learning workflow was applied to classify Tg and Wt mice. Briefly, in each classification task, the dataset was split into 80% training/validation (6 Tg, 6 Wt) and 20% testing (2 TG, 2 WT). Feature selection in the training set involved removing highly correlated features (Spearman correlation >0.8) and selecting top-ranked features based on importance scores calculated using Neighborhood Component Analysis. Various classifiers were evaluated, including logistic regression, k-nearest neighbor (KNN), linear discriminant analysis (LDA), support vector machine (SVM), multilayer perceptron neural network (MLP), random forest, and ensemble models. Hyperparameters were optimized during 3-fold cross-validation on the training/validation set. The final classifier was selected based on validation performance. The same training and testing sets were used across all parallel analyses with respect to feature types and image processing strategies. All analyses were performed using Python ver and image segmentation was done in 3D slicer ver.

### Results

Machine learning (ML) enabled 100% enabled separation of transgenic mice from wild types. To further assess subtle micro-scale probe-related changes between TG and WT mice in the in vivo post-contrast MRI data, machine learning (ML) was used, incorporating radiomic features that can quantify spatiotemporal variability, microstructural changes that conventional ROI analysis would miss. Radiomic features provide a structured, quantitative representation of biological images, and machine learning serves as the analytical framework that interprets these high-dimensional descriptors to uncover clinically meaningful patterns. Radiomics transforms MRI data into measurable features, capturing intensity distributions, spatial textures, geometric properties, and higher-order signal transformations that reflect underlying tissue biology over the entire regional volume with far greater granularity than percent change in signal in a 1.0 mm thick slab alone.

A total of 1284 RFs per region (cerebral cortex or olfactory bulb) were extracted. After feature selection, the most informative RFs were used for classification. Classifiers using olfactory bulb RFs required the use of simpler models but a larger number of features. When RFs from both the cortex and olfactory bulb were combined, the models required more features and/or exhibited poorer performance. When cortex or olfactory bulb RFs were analyzed separately, all selected RFs were derived from filtered images. As shown in Table 1, with raw images, a multi-layer perceptron (MLP) classifier with only three cortex RFs was sufficient to achieve perfect performance. Using olfactory bulb RFs allowed the model complexity to be reduced to logistic regression, albeit with a total of 10 RFs. For isotropically resampled images, a MLP classifier using three cortex RFs also achieved perfect classification, whereas a random forest classifier with 3 olfactory bulb RFs achieved the same performance. When isotropically resampled images were additionally pixel-normalized, perfect classification was achieved using a logistic regression classifier with a single cortex RF. Overall, nearly all models across the three image types and feature sets achieved perfect classification of TG mice with Parkinson's disease pathology from WT controls, with validation and testing accuracy and ROC values reaching 1.0. The only exception was the model using isotropically resampled and normalized images that combined cortex and olfactory bulb RFs (accuracy=0.75 and ROC=0.75). Notably, images resampled to isotropic resolution achieved perfect classification using the fewest features and simplest classifiers.

TABLE 1
Classification of in vivo MR images of transgenic
mice from wild types using Radiomic features

| | | Best | Validation | Validation | Testing | Testing | Number of |
|---|---|---|---|---|---|---|---|
| Image type | ROI(s) | classifier | Accuracy | ROC | Accuracy | ROC | selected RFs |
| raw | cortex | MLP | 1 | 1 | 1 | 1 | 3 |
| raw | olfactory | logistic | 1 | 1 | 1 | 1 | 10 |
| | bulb | | | | | | |
| raw | cortex and | MLP | 1 | 1 | 1 | 1 | 5 (2 from |
| | olfactory | | | | | | cortex) |
| | bulb | | | | | | |
| isotropic | cortex | MLP | 1 | 1 | 1 | 1 | 2 |
| isotropic | olfactory | Random | 1 | 1 | 1 | 1 | 3 |
| | bulb | forest | | | | | |
| isotropic | cortex and | Gaussian | 1 | 1 | 1 | 1 | 11 (5 from |
| | olfactory | naive | | | | | cortex) |
| | bulb | bayes | | | | | |
| isotropic | cortex | logistic | 1 | 1 | 1 | 1 | 1 |
| and | | | | | | | |
| normalized | | | | | | | |
| isotropic | olfactory | LDA | 1 | 1 | 1 | 1 | 4 |
| and | bulb | | | | | | |
| normalized | | | | | | | |
| isotropic | cortex and | KNN | 1 | 1 | 0.75 | 0.75 | 2 (1 from |
| and | olfactory | with k = 1 | | | | | cortex) |
| normalized | bulb | | | | | | |

LDA = linear discriminant analysis;
KNN = k-nearest neighbor;
MLP = multilayer perceptron neural network

### Example 13: Ex-Vivo Immunohistochemical (IHC) Analyses

### Mouse Brain Tissue Preparation for Microscopy

After the last MRI scan, the mice were perfused with PBS and 4% formalin, after which the brains were excised and stored in 30% sucrose solution until sectioning for histological analysis.

Anti-IBA1 staining: The brains were embedded in Tissue-Tek O.C.T. and kept in liquid nitrogen for 30 minutes. The embedded tissue was sliced into 30 m thick sections with Lecia Biosystems Cryostats at −20° C. As exemplified by the procedure for Lewy pathology, immunofluorescence studies were performed as follows: tissue sections were washed twice with PBS, followed by antigen retrieval with citric acid buffer (pH ~8.4)/microwave for 15 minutes.

The sections were washed with PBS three times and permeabilized with 0.1% Triton-X 100 for 10 minutes, followed by washing with PBS (twice). The tissue was incubated with 10% normal goat serum at room temperature for one hour, followed by incubation with a primary antibody, anti-IBA1 [Iba1/AIF-1 (E404W) XP® Rabbit mAb #17198, Cell Signaling Technology] (1:200 in 1% goat serum) and purified anti-β-amyloid [purified anti-β-amyloid 17-24 antibody, (4G8), host: mouse, Biolegend] (1:200 in 1% goat serum) overnight at 4° C. The tissue was washed with PBS three times and incubated for two hours at room temperature with Alexa Fluor 488 (anti-mouse) and Alexa Fluor 750 (anti-rabbit) secondary antibody (1:200 in PBS). After being washed with PBS two times, the tissue was incubated with Hoechst (33342, Thermo Fisher Scientific) at room temperature for 7 minutes. The sections were washed with PBS three times, coverslipped, and imaged under an Olympus IX81 microscope using standard excitation/emission filters.

GFAP staining: The brains were embedded in Tissue-Tek O.C.T. and kept in liquid nitrogen for 30 minutes. The embedded tissue was sliced into 30 m thick sections with Lecia Biosystems Cryostats at −20° C. As exemplified by the procedure for Lewy pathology, immunofluorescence studies were performed as follows: tissue sections were washed twice with PBS, followed by antigen retrieval with citric acid buffer (pH ~8.4)/microwave for 15 minutes. The sections were then washed with PBS three times and permeabilized with 0.1% Triton-X 100 for 10 minutes, followed by washing with PBS (twice). The tissue was incubated with 10% normal goat serum at room temperature for one hour, followed by incubation with a primary antibody, anti-GFAP [Anti-GFAP Mouse Monoclonal Antibody clone: 2E1.E9, Cell Signaling Technology](1:200 in 1% goat serum) and purified anti-β-amyloid [purified anti-β-amyloid 17-24 antibody, (4G8), host: mouse, Biolegend] (1:200 in 1% goat serum) overnight at 4° C. The tissue was washed with PBS three times and incubated for two hours at room temperature with Alexa Fluor 488 (anti-mouse) and Alexa Fluor 750 (anti-rabbit) secondary antibody (1:200 in PBS). After being washed with PBS two times, the tissue was incubated with Hoechst (33342, Thermo Fisher Scientific) at room temperature for 7 minutes. The sections were washed with PBS three times, coverslipped, and imaged under an Olympus IX81 microscope using standard excitation/emission filters.

### Statistical Analyses

Statistical analysis for the in vitro cell experiments was performed through one-way ANOVA with multiple pairwise comparisons between groups using Fisher's least significant difference (LSD) test. Statistical analysis of in vivo experimental data was performed through the Kruskal-Wallis method with multiple pairwise comparisons using the Bonferroni method.

### Results

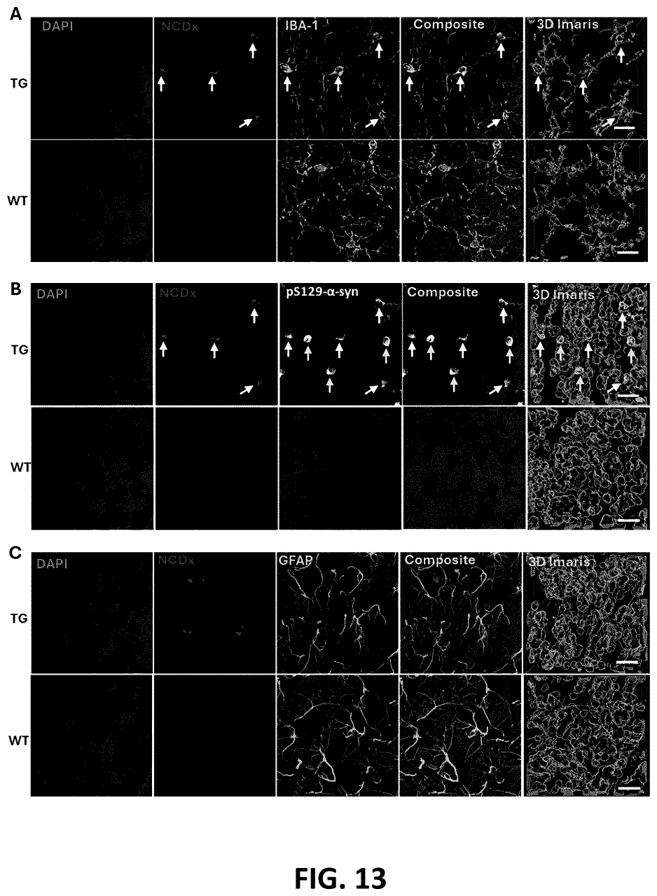

NCDx identifies disease through labeling of microglial in vivo. To confirm that the observed increase in signal intensity from the baseline in TG mice treated with NCDx was due to microglia labeling by the agent, brain tissue from the treated mice and WT controls was subjected to ex-vivo IHC studies. The presence of NCDx in the tissue was tracked using the fluorescent Dil dye incorporated in the nanoparticle formulation stage, Lewy pathology by antibody staining the tissue for α-syn phosphorylation at serine 129 (pS129-α-syn, pathologic α-syn) activity, ionized calcium-binding adaptor molecule 1 (IBA-1) activity, and glial fibrillary acidic protein (GFAP) for astrocyte activity. As exemplified by the confocal microscopy images in FIG. 13, tissue sections from TG mice stained with DAPI (nuclei stain) and anti-IBA-1 antibody (FIG. 13(A), top panel), revealed focal structures with Dil fluorescence (NCDx) confirming the presence of NCDx in the tissue and IBA-1 reactivity. A composite image formed by merging the three signals showed colocalization of the NCDx and IBA-1 signals (white arrows). 3D Imaris images generated by merging the NCDx and IBA-1 signals showed that the NCDx signal was cytosolic. Tissue sections from the WT controls (bottom panel) showed IBA-1 reactivity but no significant organized NCDx signal, suggesting no significant uptake of NCDx by microglia in the WT brain, consistent with the in vivo MRI data.

To ascertain that the observer microglial reactivity correlated with the presence of Lewy pathology, the same tissue sections was further stained with anti-pS129-α-syn antibody. As shown in the representative images in FIG. 13(B) (top panel), apart from the NCDx signal, well organized structures (non-white arrows) attributed to Lewy bodies as well as some less organized pS129-α-syn reactive structures (white arrows) were also observed. A composite image formed by merging the three images showed co-localization of the diffuse Lewy pathology with the NCDx signal. 3D Imaris image of the composite suggest that the diffuse Lewy pathology was within the cytosol of the IBA-1 reactive cells. Images from WT controls (bottom panel) showed no observable pS129-α-syn reactivity as observed in the TG brain tissue. Contiguous sections from both TG and WT mice were further stained with anti-GFAP antibody to assess any meaningful correlation between NCDx and astrocyte activity.

As summarized in FIG. 13(C), TG tissue sections showed both NCDx signal and GFAP reactivity, but a composite image formed by merging both signals with the DAPI signal showed no meaningful overlap between the two signals, even in 3D Imaris images. Tissue sections from WT controls showed GFAP reactivity but no significant NCDx signal as expected.

This model develops α-syn aggregates, which originate from the olfactory bulb and spinal cord, and then spread to the brainstem, cerebellum, midbrain, and cortical areas, in a manner that parallels the progression of the disease in humans.

### Example 14: Preparation of HLNT-EtOH

### Synthesis of Compound 13

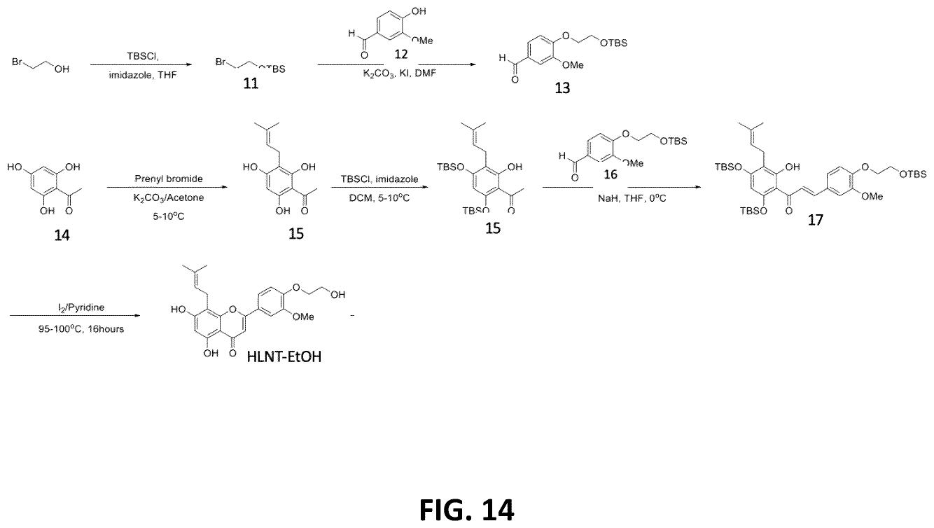

Referring to FIG. 14, Vanillin (30.0 g, 0.19 mol), potassium carbonate (52.4 g, 0.38 mol), and potassium iodide (31.54 g, 0.19 mol) were suspended in DMF (300 ml), followed by slow addition of compound 11 (71.7 g, 0.3 mol) in DMF (600 mL). The resultant mixture was stirred at 80° C. for 20 h. Progress of the reaction was monitored by TLC. After consumption of starting material (by TLC), the reaction mass was quenched with ice cold water (1500 mL), and the product was extracted with ethyl acetate (3*1000 mL). The organic layer was washed with 1M NaCl (3*1000 mL) and dried under reduced pressure to obtain compound 13 (51 g, yield 85%).

### Synthesis of Compound 15

Compound 14 (25 g, 0.148 mol) was dissolved in acetone (500 mL) followed by addition of K₂CO₃ (20.4 g, 0.148 mol) and prenyl bromide (15.4. g, 0.103 mol) at 0-5° C. The resulting mixture was stirred at the same temperature for 2 hours followed by filtration of solids and concentration under reduced pressure to obtain the crude product, which was further purified by column chromatography eluted with ethyl acetate/hexanes gradient to afford the desired product 15 (10.9 g, yield 31%) as a white solid.

### Synthesis of Compound 16

Compound 15 (30.0 g, 0.127 mol) was dissolved in DCM (300 mL) followed by addition of imidazole (34.54 g, 0.51 mol) at 0-5° C. TBSCl (57.53 g, 0.38 mol) was added at 5-10° C. dropwise, and the reaction temperature was allowed to slowly warm to room temperature over 3 hours. After consumption of starting material (by TLC), the reaction mass was quenched with ice cold water (200 mL), and the product was extracted with dichloromethane (3*200 mL). The organic layer was washed with 1M NaHCO₃ (3*200 mL) and evaporated under reduced pressure to obtain a residue, which was purified by column chromatography eluted with ethyl acetate/hexanes gradient to afford the desired product 16 (52 g, yield 88%) as a yellow oil.

### Synthesis of Compound 17

Compound 16 (30 g, 64.52 mmol) was dissolved in THF (300 mL) at 0-5° C. followed by slow addition of NaH (3.87 g, 96.77 mmol) and stirred for 10-15 min. This was followed by addition of compound 13 (30 g, 96.77 mmol) in THF (300 mL) dropwise at 5-10° C. Progress of the reaction was monitored by TLC. After consumption of starting materials (by TLC), the reaction mass was quenched with ammonium chloride (400 mL) at 0° C., and the product was extracted with ethyl acetate (3*300 mL). The organics layer was evaporated, and the residue was purified by column chromatography eluted with ethyl acetate/hexanes gradient to afford the desired product 17 (37.6 g, yield 77%) as yellow oil.

### Synthesis of HLNT-EtOH

Compound 17 (1.0 g, 1.32 mmol) was suspended in 100 mL of pyridine followed by addition of Iodine (0.4 g, 1.59 mmol) and heated at 95-100° C. for 16 h. The ensuing mixture was cooled to room temperature, diluted with water, and the pH adjusted to 3-4 with citric acid. The mixture was further diluted with 10% Na₂S₂O₃ solution, extracted with EtOAc, washed with water (2*100 mL), dried over sodium sulphate, and evaporated the solvent removed under reduced pressure to get crude product as yellow colored solid. The residue was precipitated in ethyl acetate and filtered to afford the desired product HLNT-EtOH (0.39 g, yield 30%) as a yellow solid.

### Example 14: Synthesis DSPE-PEG3400-Caflanone v.4

### Synthesis of Compound 18

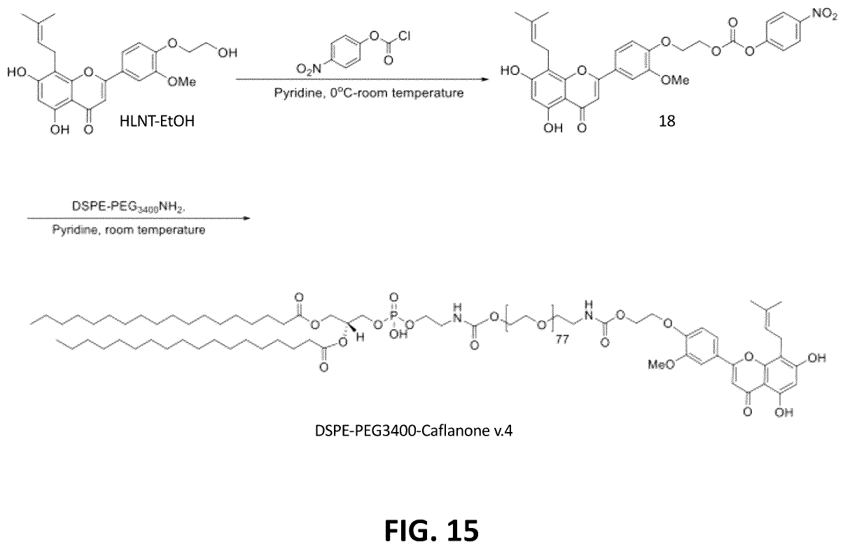

Referring to FIG. 15, HLNT-EtOH (300 mg, 0.72 mmol) was suspended in dry pyridine (10 ml) at 0-5° C. followed by slow addition of 4-nitrophenyl chloroformate (584 mg, 2.91 mmol) in dichloromethane (1 mL). The resultant mixture was allowed to slowly warm to room temperature and was stirred for 3 h and quenched with ice cold water (10 mL). The product was extracted with ethyl acetate (3*20 mL), washed with 1M NaHCO3 (3*10 mL) and then 1M HCl (3*10 mL). The ethyl acetate layer was evaporated under reduced pressure, and the residue purified by column chromatography eluted with ethyl acetate/hexanes gradient to afford the desired product 18 (270 mg, yield 64%) as a yellow solid.

Compound 18 (250 mg, 0.43 mmol) was suspended in dry pyridine (10 ml) at 0-5° C. To this was added DSPE-PEG₃₄₀₀-NH₂ (360 mg, 0.08 mmol) in dichloromethane (1 mL). The resultant mixture was slowly raised to room temperature and continued for 6 hours, after which it was concentrated under reduced pressure. The residue was diluted with methanol/water mixture (1:1, 8 ml) and loaded into a 2000 MWCO dialysis bag and dialyzed against MES buffer (50 mM, 2×5 liters) for 8 hours and then water (3×5 liters) for 2 days. The water was removed by freeze drying to obtain DSPE-PEG3400-Caflanone v.4 as a yellow solid (190 mg, 51%).

### Example 15: Macrophage Cell Uptake Studies Using DSPE-PEG3400-Caflanone v.4

RAW264.7 murine macrophages were used for a cell uptake and kinetics study. Cells were seeded at a density of 5,000 cells per well in PerkinElmer PhenoPlate 96-well plates and allowed to adhere overnight at 37° C. in a humidified incubator with 5% CO₂. The cells were treated with 25 ng/mL colony-stimulating factor-1 (CSF1) for 24 hours, as described by Morandi, A., Barbetti, V., Riverso, M., Dello Sbarba, P., Rovida, E., 2011. The Colony-Stimulating Factor-1 (CSF-1) Receptor Sustains ERK1/2 Activation and Proliferation in Breast Cancer Cell Lines. PLoS ONE 6, e27450. https://doi.org/10.1371/journal.pone.0027450.

After CSF1 stimulation, cells were treated with 80 μM NCDx nanoparticles or non-targeted nanoparticles for 2 hours. For uptake kinetics studies, cells were incubated with nanoparticles and fixed at 15-minute intervals (TO-T120 min). At each time point, cells were washed twice with 1×PBS and fixed using 10% formalin with 0.2% Triton X-100 for 10 minutes, followed by three washes with PBS.

To visualize the cytoskeleton, cells were stained with CellMask™ Actin-488 (Thermo Fisher Scientific, USA), and nuclei were counterstained with Hoechst dye. Imaging was performed with an Olympus IX81 fluorescence microscope using a 40×objective. The formula used to calculate ET50 was ET50=T0+0.5*(Tmax−Tmin).

### Results

In cellular uptake studies, the non-targeted nanoparticle group (NT) showed minimal to no detectable NCDx signal. In contrast, CSF1-treated RAW264.7 macrophages displayed a clear and specific uptake of targeted NCDx nanoparticles, indicating receptor-mediated internalization.

For kinetic analysis, the uptake of NCDx nanoparticles was measured overtime from TO to T120 min at 15-minute intervals. A detectable uptake signal appeared as early as 15 minutes, with a significant increase from 30 to 45 minutes. Uptake reached a plateau at about 90 minutes, indicating saturation of internalization. The estimated ET50 value for NCDx nanoparticle uptake in RAW264.7 macrophages was around 53 minutes.

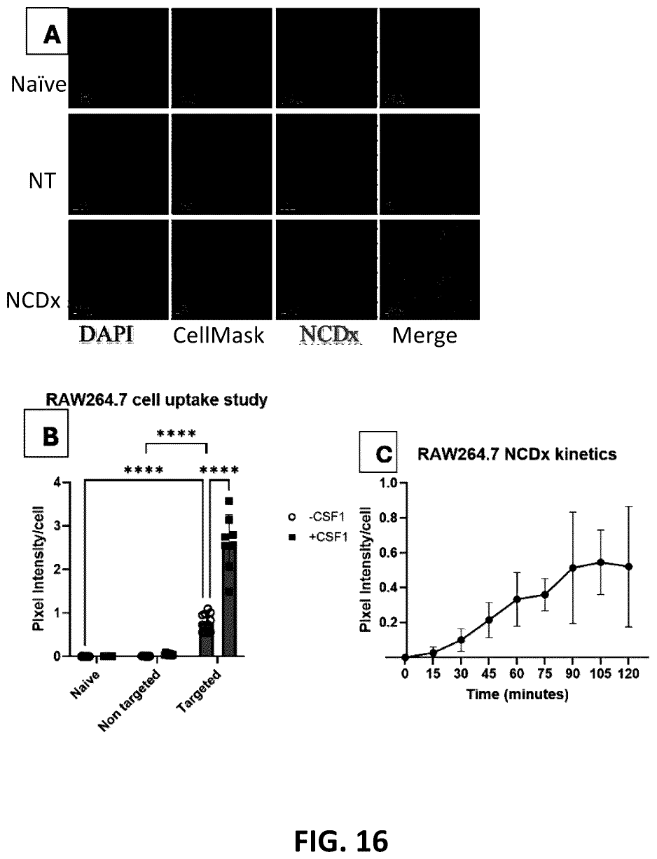

Notably, CSF1 stimulation significantly improved nanoparticle uptake, as shown in FIG. 16(B). In contrast, non-targeted nanoparticles had uptake levels similar to naïve controls, indicating negligible uptake within the 2-hour incubation period.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

1. A liposomal composition, the liposomal composition comprising:
a first phospholipid;
a sterically bulky excipient that is capable of stabilizing the liposomal composition;
a second phospholipid that is derivatized with a first polymer;
a macrocyclic gadolinium-based imaging agent;
and a third phospholipid that is derivatized with a second polymer, the second polymer being conjugated to a targeting ligand, the targeting ligand being represented by: 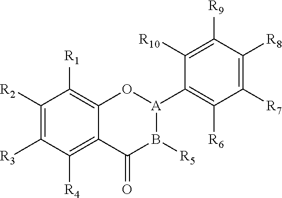
wherein R₁-R₁₀ may be any one or more substituents selected from a hydrogen molecule (H), a hydroxide molecule (OH), a methyl group (CH3), a C₁-C₆ alkoxy group, a carboxyl group (COOH), a halide, an amino acid, a C₁-C₆ alkyl or alkenyl, or a C₁-C₆ alkyl amine, with the caveat that one of R₁ or R₃ must be either 3-methyl-2-butenyl or 3,7-dimethyl-2,6-octenyl, and the carbon-to-carbon A and B bond is either a single flavanone bond or a double flavone bond, or a salt thereof.

2. The liposomal composition of claim 1, wherein the targeting ligand comprises: 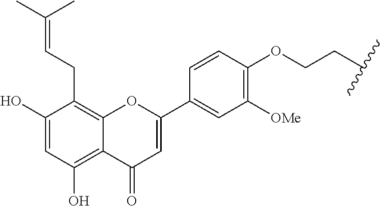 [SMILES: *CCOc1ccc(-c2cc(=O)c3c(O)cc(O)c(CC=C(C)C)c3o2)cc1OC]
or a salt thereof.

3. The liposomal composition of claim 1, wherein the targeting ligand is: 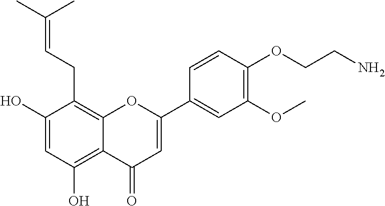 [SMILES: COc1cc(-c2cc(=O)c3c(O)cc(O)c(CC=C(C)C)c3o2)ccc1OCCN]
or a salt thereof.

4. The liposomal composition of claim 1, wherein the targeting ligand is: 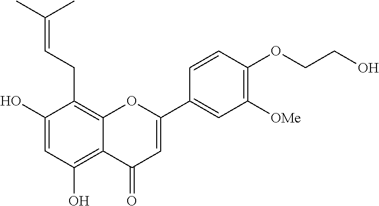 [SMILES: COc1cc(-c2cc(=O)c3c(O)cc(O)c(CC=C(C)C)c3o2)ccc1OCCO]
or a salt thereof.

5. The liposomal composition of claim 1, wherein the first phospholipid comprises hydrogenated soy L-α-phosphatidylcholine (“HSPC”).

6. The liposomal composition of claim 1, wherein the sterically bulky excipient that is capable of stabilizing the liposomal composition comprises cholesterol.

7. The liposomal composition of claim 1, wherein the second phospholipid that is derivatized with a first polymer comprises 1,2-distearoyl-sn-glycero-3-phosphoethanolamine-N-(methoxy (polyethylene glycol)-2000).

8. The liposomal composition of claim 1, wherein the macrocyclic gadolinium-based imaging agent comprises gadolinium(3+) 2-[4,7,10-tris(carboxylatomethyl)-1,4,7,10-tetrazacyclododec-1-yl]acetate and is conjugated to a fourth phospholipid as represented by the structure: 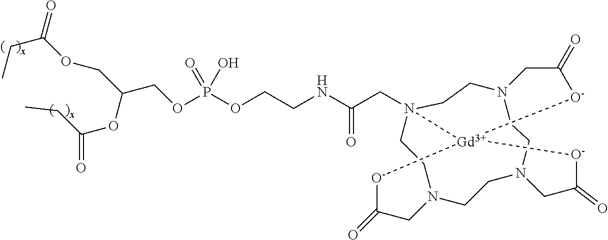
or a salt thereof, wherein the variable x may be one of: 12, 13, 14, 15, 16, 17, or 18.

9. The liposomal composition of claim 1, wherein the third phospholipid that is derivatized with a second polymer comprises: 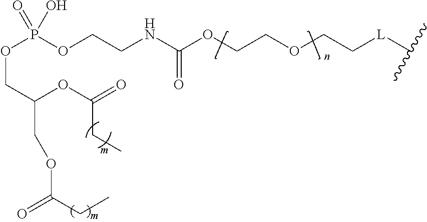
or a salt thereof, wherein the variable n may be any integer from about 60 to about 100, the variable m may be one of: 12, 13, 14, 15, 16, 17, or 18, and L is a linker having an amido group, an ether group, or both.

10. The liposomal composition of claim 9, wherein the variable n is 77 and the variable m is 16.

11. The liposomal composition of claim 2, wherein the third phospholipid that is derivatized with a second polymer, the second polymer being conjugated to the targeting ligand is: 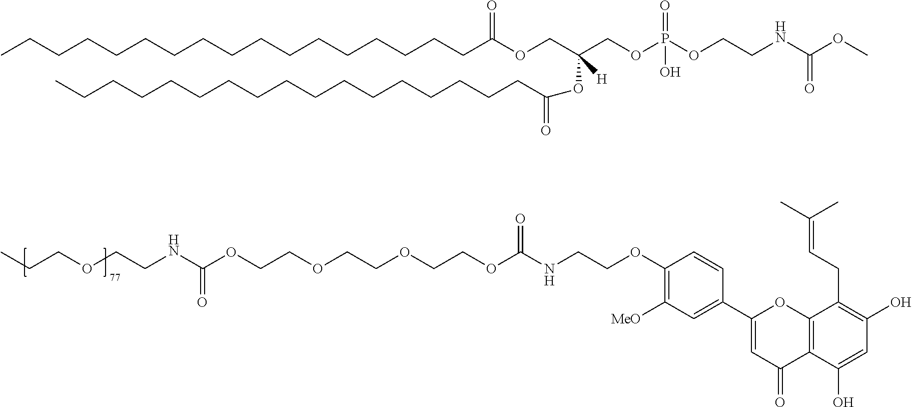
or a salt thereof.

12. The liposomal composition of claim 2, wherein the third phospholipid that is derivatized with a second polymer, the second polymer being conjugated to the targeting ligand is: 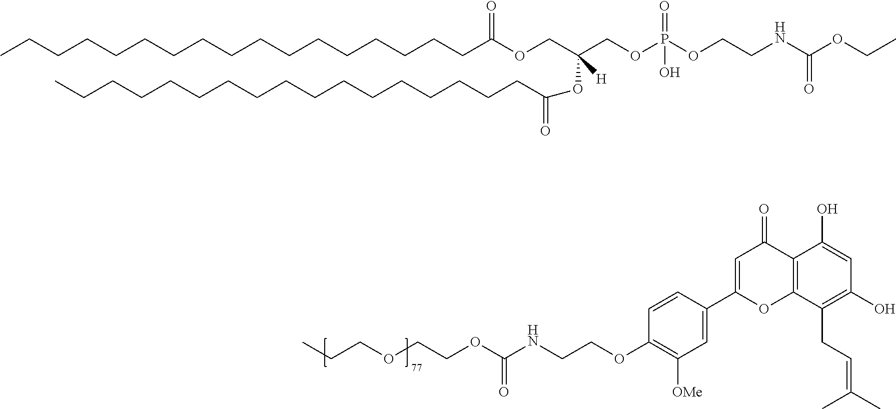
or a salt thereof.

13. The liposomal composition of claim 2, wherein the third phospholipid that is derivatized with a second polymer, the second polymer being conjugated to the targeting ligand is: 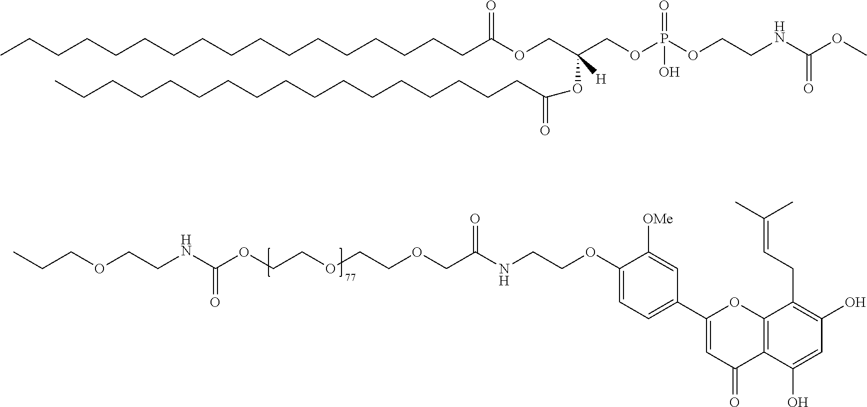
or a salt thereof.

14. The liposomal composition of claim 2, wherein the third phospholipid that is derivatized with a second polymer, the second polymer being conjugated to the targeting ligand is: 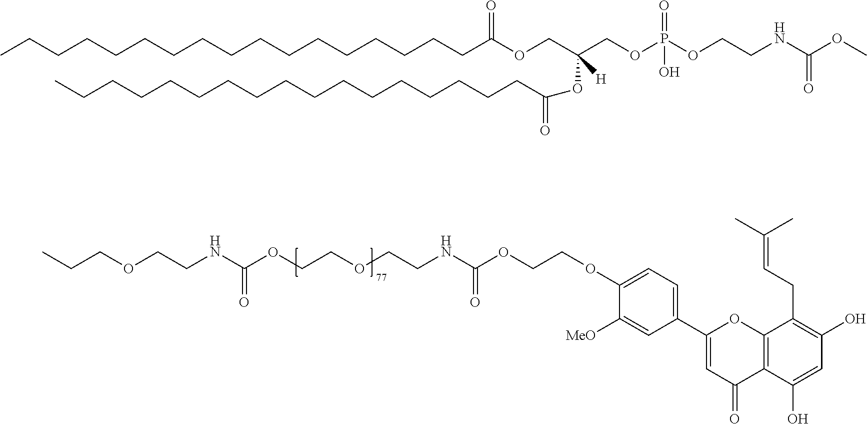
or a salt thereof.

15. A method for MRI imaging activated microglia cells in a subject, the method comprising introducing in the subject a detectable quantity of the liposomal composition according to any one of claims 2 or 11-14; allowing sufficient time for the liposomal composition to be associated with the activated microglia cells; and detecting the liposomal composition associated with the activated microglia cells.

16. The method of claim 15, wherein the activated microglia cells are associated with cross-β sheet deposits.

17. The method of claim 16, wherein the cross-β sheet deposits comprise one or more of α-synuclein, amyloid-β, tau, TDP43, TTR, and HTT.

18. A targeting ligand comprising: 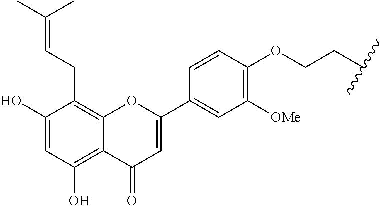 [SMILES: *CCOc1ccc(-c2cc(=O)c3c(O)cc(O)c(CC=C(C)C)c3o2)cc1OC]
or a salt thereof.

19. The targeting ligand of claim 18, wherein the targeting ligand is: 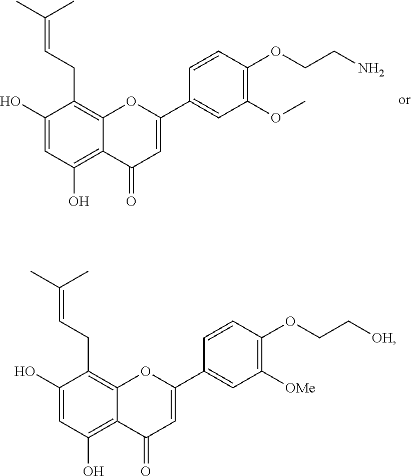 [SMILES: COc1cc(-c2cc(=O)c3c(O)cc(O)c(CC=C(C)C)c3o2)ccc1OCCN ; COc1cc(-c2cc(=O)c3c(O)cc(O)c(CC=C(C)C)c3o2)ccc1OCCO]
or a salt thereof.

20. A method for MRI imaging peripheral inflammation due to activated macrophages in a subject, the method comprising introducing in the subject a detectable quantity of the liposomal composition according to any one of claims 2 or 11-14; allowing sufficient time for the liposomal composition to be associated with the peripheral inflammation; and detecting the liposomal composition associated with the peripheral inflammation.
